SEQUENCE LISTING (71 sequences)

SEQ ID NO: 1 - DNA, 19 bp, synthetic construct
  misc_feature 1..19: note = VH3-1-f
  source 1..19: mol_type = other DNA
ctccggattcactttcagc

SEQ ID NO: 2 - DNA, 18 bp, synthetic construct
  misc_feature 1..18: note = VH3-1-b
  source 1..18: mol_type = other DNA
ttacctggtgcctgtctg

SEQ ID NO: 3 - DNA, 18 bp, synthetic construct
  misc_feature 1..18: note = VH3-2-f
  source 1..18: mol_type = other DNA
ggactggagtgggtctct

SEQ ID NO: 4 - DNA, 18 bp, synthetic construct
  misc_feature 1..18: note = VH3-2-b
  source 1..18: mol_type = other DNA
gcgtgagatggtgaagcg

SEQ ID NO: 5 - DNA, 18 bp, synthetic construct
  misc_feature 1..18: note = lib-cdr-f
  source 1..18: mol_type = other DNA
gtcagtcacgctctaagg

SEQ ID NO: 6 - DNA, 18 bp, synthetic construct
  misc_feature 1..18: note = lib-cdr-b
  source 1..18: mol_type = other DNA
ctgagtcgatgacctacg

SEQ ID NO: 7 - DNA, 19 bp, synthetic construct
  misc_feature 1..19: note = VH-3-f
  source 1..19: mol_type = other DNA
cactgccgtgtattactgc

SEQ ID NO: 8 - DNA, 18 bp, synthetic construct
  misc_feature 1..18: note = VH-3-b
  source 1..18: mol_type = other DNA
cagagtaccttgtcccca

SEQ ID NO: 9 - DNA, 18 bp, synthetic construct
  misc_feature 1..18: note = VL-1-f
  source 1..18: mol_type = other DNA
cgcgtcaccatcagctgc

SEQ ID NO: 10 - DNA, 19 bp, synthetic construct
  misc_feature 1..19: note = VL-1-b
  source 1..19: mol_type = other DNA
ctgggagttgctgatacca

SEQ ID NO: 11 - DNA, 20 bp, synthetic construct
  misc_feature 1..20: note = VL-2-f
  source 1..20: mol_type = other DNA
ctcctaagctcctgatttac

SEQ ID NO: 12 - DNA, 18 bp, synthetic construct
  misc_feature 1..18: note = VL-2-b
  source 1..18: mol_type = other DNA
aaagcgatcaggcacacc

SEQ ID NO: 13 - DNA, 19 bp, synthetic construct
  misc_feature 1..19: note = VL-3-f
  source 1..19: mol_type = other DNA
cgaggctgactattactgc

SEQ ID NO: 14 - DNA, 18 bp, synthetic construct
  misc_feature 1..18: note = VL-3-b
  source 1..18: mol_type = other DNA
agtttggtcccaccgccg

SEQ ID NO: 15 - DNA, 18 bp, synthetic construct
  misc_feature 1..18: note = VK-1-f
  source 1..18: mol_type = other DNA
cgcgcaactctgtcttgt

SEQ ID NO: 16 - DNA, 20 bp, synthetic construct
  misc_feature 1..20: note = VK-1-b
  source 1..20: mol_type = other DNA
ccaggtttctgttggtacca

SEQ ID NO: 17 - DNA, 18 bp, synthetic construct
  misc_feature 1..18: note = VK-2-f
  source 1..18: mol_type = other DNA
ccacgcctgctcatctat

SEQ ID NO: 18 - DNA, 18 bp, synthetic construct
  misc_feature 1..18: note = VK-2-b
  source 1..18: mol_type = other DNA
gaacctgtctgggatgcc

SEQ ID NO: 19 - DNA, 21 bp, synthetic construct
  misc_feature 1..21: note = VK-3-f
  source 1..21: mol_type = other DNA
gacttcgcagtttactattgt

SEQ ID NO: 20 - DNA, 18 bp, synthetic construct
  misc_feature 1..18: note = VK-3-b
  source 1..18: mol_type = other DNA
accttcgttccctgacca

SEQ ID NO: 21 - DNA, 18 bp, synthetic construct
  misc_feature 1..18: note = pUC57-b
  source 1..18: mol_type = other DNA
ttcgccattcaggctgcg

SEQ ID NO: 22 - DNA, 19 bp, synthetic construct
  misc_feature 1..19: note = VH3-1-f-rc
  source 1..19: mol_type = other DNA
gctgaaagtgaatccggag

SEQ ID NO: 23 - DNA, 18 bp, synthetic construct
  misc_feature 1..18: note = VH3-2-f-rc
  source 1..18: mol_type = other DNA
agagacccactccagtcc

SEQ ID NO: 24 - DNA, 19 bp, synthetic construct
  misc_feature 1..19: note = VH-3-rc-F
  source 1..19: mol_type = other DNA
gcagtaatacacggcagtg

SEQ ID NO: 25 - DNA, 18 bp, synthetic construct
  misc_feature 1..18: note = VL-1-f-rc
  source 1..18: mol_type = other DNA
gcagctgatggtgacgcg

SEQ ID NO: 26 - DNA, 20 bp, synthetic construct
  misc_feature 1..20: note = VL-2-f-rc
  source 1..20: mol_type = other DNA
gtaaatcaggagcttaggag

SEQ ID NO: 27 - DNA, 19 bp, synthetic construct
  misc_feature 1..19: note = VL-3-f-rc
  source 1..19: mol_type = other DNA
gcagtaatagtcagcctcg

SEQ ID NO: 28 - DNA, 18 bp, synthetic construct
  misc_feature 1..18: note = VK-1-f-rc
  source 1..18: mol_type = other DNA
acaagacagagttgcgcg

SEQ ID NO: 29 - DNA, 18 bp, synthetic construct
  misc_feature 1..18: note = VK-2-f-rc
  source 1..18: mol_type = other DNA
atagatgagcaggcgtgg

SEQ ID NO: 30 - DNA, 21 bp, synthetic construct
  misc_feature 1..21: note = VK-3-f-rc
  source 1..21: mol_type = other DNA
acaatagtaaactgcgaagtc

SEQ ID NO: 31 - DNA, 18 bp, synthetic construct
  misc_feature 1..18: note = hCH2-b
  source 1..18: mol_type = other DNA
cttgacctcagggtcttc

SEQ ID NO: 32 - DNA, 18 bp, synthetic construct
  misc_feature 1..18: note = VH3-1-b-rc
  source 1..18: mol_type = other DNA
cagacaggcaccaggtaa

SEQ ID NO: 33 - DNA, 18 bp, synthetic construct
  misc_feature 1..18: note = VH3-2-b-rc
  source 1..18: mol_type = other DNA
cgcttcaccatctcacgc

SEQ ID NO: 34 - DNA, 18 bp, synthetic construct
  misc_feature 1..18: note = VH-3-rc-B
  source 1..18: mol_type = other DNA
tggggacaaggtactctg

SEQ ID NO: 35 - DNA, 19 bp, synthetic construct
  misc_feature 1..19: note = VL-1-b-rc
  source 1..19: mol_type = other DNA
tggtatcagcaactcccag

SEQ ID NO: 36 - DNA, 18 bp, synthetic construct
  misc_feature 1..18: note = VL-2-b-rc
  source 1..18: mol_type = other DNA
ggtgtgcctgatcgcttt

SEQ ID NO: 37 - DNA, 18 bp, synthetic construct
  misc_feature 1..18: note = VL-3-b-rc
  source 1..18: mol_type = other DNA
cggcggtgggaccaaact

SEQ ID NO: 38 - DNA, 20 bp, synthetic construct
  misc_feature 1..20: note = VK-1-b-rc
  source 1..20: mol_type = other DNA
tggtaccaacagaaacctgg

SEQ ID NO: 39 - DNA, 18 bp, synthetic construct
  misc_feature 1..18: note = VK-2-b-rc
  source 1..18: mol_type = other DNA
ggcatcccagacaggttc

SEQ ID NO: 40 - DNA, 18 bp, synthetic construct
  misc_feature 1..18: note = VK-3-b-rc
  source 1..18: mol_type = other DNA
tggtcagggaacgaaggt

SEQ ID NO: 41 - DNA, 18 bp, synthetic construct
  misc_feature 1..18: note = pUC57-in-b
  source 1..18: mol_type = other DNA
ggatgtgctgcaaggcga

SEQ ID NO: 42 - DNA, 18 bp, synthetic construct
  misc_feature 1..18: note = hCH2-in-b
  source 1..18: mol_type = other DNA
ccaggagttcaggtgctg

SEQ ID NO: 43 - DNA, 19 bp, synthetic construct
  misc_feature 1..19: note = pC3x-f
  source 1..19: mol_type = other DNA
gcacgacaggtttcccgac

SEQ ID NO: 44 - DNA, 18 bp, synthetic construct
  misc_feature 1..18: note = VH3-1-b
  source 1..18: mol_type = other DNA
ttacctggtgcctgtctg

SEQ ID NO: 45 - DNA, 18 bp, synthetic construct
  misc_feature 1..18: note = VH3-1-b-rc
  source 1..18: mol_type = other DNA
cagacaggcaccaggtaa

SEQ ID NO: 46 - DNA, 18 bp, synthetic construct
  misc_feature 1..18: note = VH3-2-b
  source 1..18: mol_type = other DNA
gcgtgagatggtgaagcg

SEQ ID NO: 47 - DNA, 18 bp, synthetic construct
  misc_feature 1..18: note = VH3-2-b-rc
  source 1..18: mol_type = other DNA
cgcttcaccatctcacgc

SEQ ID NO: 48 - DNA, 18 bp, synthetic construct
  misc_feature 1..18: note = VH-3-b
  source 1..18: mol_type = other DNA
cagagtaccttgtcccca

SEQ ID NO: 49 - DNA, 18 bp, synthetic construct
  misc_feature 1..18: note = VH-3-rc-B
  source 1..18: mol_type = other DNA
tggggacaaggtactctg

SEQ ID NO: 50 - DNA, 20 bp, synthetic construct
  misc_feature 1..20: note = VL-2-f-rc
  source 1..20: mol_type = other DNA
gtaaatcaggagcttaggag

SEQ ID NO: 51 - DNA, 20 bp, synthetic construct
  misc_feature 1..20: note = VL-2-f
  source 1..20: mol_type = other DNA
ctcctaagctcctgatttac

SEQ ID NO: 52 - DNA, 19 bp, synthetic construct
  misc_feature 1..19: note = VL-3-f-rc
  source 1..19: mol_type = other DNA
gcagtaatagtcagcctcg

SEQ ID NO: 53 - DNA, 19 bp, synthetic construct
  misc_feature 1..19: note = VL-3-f
  source 1..19: mol_type = other DNA
cgaggctgactattactgc

SEQ ID NO: 54 - DNA, 20 bp, synthetic construct
  misc_feature 1..20: note = pC3x-b
  source 1..20: mol_type = other DNA
aaccatcgatagcagcaccg

SEQ ID NO: 55 - DNA, 18 bp, synthetic construct
  misc_feature 1..18: note = VH-3-rc-B
  source 1..18: mol_type = other DNA
tggggacaaggtactctg

SEQ ID NO: 56 - DNA, 18 bp, synthetic construct
  misc_feature 1..18: note = VK-2-f-rc
  source 1..18: mol_type = other DNA
atagatgagcaggcgtgg

SEQ ID NO: 57 - DNA, 18 bp, synthetic construct
  misc_feature 1..18: note = VK-2-f
  source 1..18: mol_type = other DNA
ccacgcctgctcatctat

SEQ ID NO: 58 - DNA, 21 bp, synthetic construct
  misc_feature 1..21: note = VK-3-f-rc
  source 1..21: mol_type = other DNA
acaatagtaaactgcgaagtc

SEQ ID NO: 59 - DNA, 21 bp, synthetic construct
  misc_feature 1..21: note = VK-3-f
  source 1..21: mol_type = other DNA
gacttcgcagtttactattgt

SEQ ID NO: 60 - DNA, 20 bp, synthetic construct
  misc_feature 1..20: note = pC3x-b
  source 1..20: mol_type = other DNA
aaccatcgatagcagcaccg

SEQ ID NO: 61 - DNA, 19 bp, synthetic construct
  misc_feature 1..19: note = pC3x-f
  source 1..19: mol_type = other DNA
gcacgacaggtttcccgac

SEQ ID NO: 62 - DNA, 18 bp, synthetic construct
  misc_feature 1..18: note = VH-3-b
  source 1..18: mol_type = other DNA
cagagtaccttgtcccca

SEQ ID NO: 63 - DNA, 18 bp, synthetic construct
  misc_feature 1..18: note = VH-3-rc-B
  source 1..18: mol_type = other DNA
tggggacaaggtactctg

SEQ ID NO: 64 - DNA, 20 bp, synthetic construct
  misc_feature 1..20: note = pC3x-b
  source 1..20: mol_type = other DNA
aaccatcgatagcagcaccg

SEQ ID NO: 65 - DNA, 18 bp, synthetic construct
  misc_feature 1..18: note = VH-3-rc-B
  source 1..18: mol_type = other DNA
tggggacaaggtactctg

SEQ ID NO: 66 - DNA, 20 bp, synthetic construct
  misc_feature 1..20: note = pC3x-b
  source 1..20: mol_type = other DNA
aaccatcgatagcagcaccg

SEQ ID NO: 67 - DNA, 19 bp, synthetic construct
  misc_feature 1..19: note = pC3x-f
  source 1..19: mol_type = other DNA
gcacgacaggtttcccgac

SEQ ID NO: 68 - DNA, 20 bp, synthetic construct
  misc_feature 1..20: note = pC3x-b
  source 1..20: mol_type = other DNA
aaccatcgatagcagcaccg

SEQ ID NO: 69 - DNA, 22 bp, synthetic construct
  misc_feature 1..22: note = omp seq
  source 1..22: mol_type = other DNA
aagacagctatcgcgattgcag

SEQ ID NO: 70 - protein, 211 aa, synthetic construct
  REGION 1..211: note = (IGHV3-23)-linker-(IGKV3-20)
EVQLLESGGGLVQPGGSLRLSCAASGFTFSXXXXXWVRQAPGKGLEWVXXXXXRFTISRDNSKNTLYLQMNSLRAEDTAVYYCAKXXXXXWGQGTLVTVSSGGGGSGGGGSGGGGSEIVLTQSPGTLSLSPGERATLSCXXXXXWYQQKPGQAPRLLIYXXXXXGIPDRFSGSGSGTDFTLTISRLEPEDFAVYYCXXXXXFGQGTKVEIK

SEQ ID NO: 71 - protein, 210 aa, synthetic construct
  REGION 1..210: note = (IGHV3-23)-linker-(IGLV1-47)
EVQLLESGGGLVQPGGSLRLSCAASGFTFSXXXXXWVRQAPGKGLEWVXXXXXRFTISRDNSKNTLYLQMNSLRAEDTAVYYCAKXXXXXWGQGTLVTVSSGGGGSGGGGSGGGGSQSVLTQPPSASGTPGQRVTISCXXXXXWYQQLPGTAPKLLIYXXXXXGVPDRFSGSKSGTSASLAISGLRSEDEADYYCXXXXXFGGGTKLTVL

(19) United States
(12) Patent Application Publication
(10) Pub. No.: US 2023/0332141 A1
(43) Pub. Date: Oct. 19, 2023

(54) NOVEL ANTIBODY LIBRARY PREPARATION METHOD AND LIBRARY PREPARED THEREBY

(71) Applicant: EWHA UNIVERSITY-INDUSTRY COLLABORATION FOUNDATION, Seoul (KR)

(72) Inventors: Hyunbo SHIM, Seoul (KR); Xuelian BAI, Yanji City (CN); Moonseon JANG, Goyang-si (KR)

(21) Appl. No.: 18/173,821

(22) Filed: Feb. 24, 2023

Related U.S. Application Data
(63) Continuation of application No. PCT/KR2021/011388, filed on Aug. 25, 2021.

(30) Foreign Application Priority Data
Aug. 26, 2020 (KR) 10-2020-0107932

(51) Int. Cl. C12N 15/10; C07K 16/00

(52) U.S. Cl. CPC C12N 15/1089; C07K 16/005; C07K 2317/565; C07K 2317/567

(57) ABSTRACT

The present disclosure relates to a novel method of preparing an antibody library and the prepared library therefrom. The antibody library prepared by the preparation method according to an embodiment of the present disclosure contains antibodies having excellent physical properties to a large number of antigens, and thus can be favorably used as an antibody library that has functional diversity, contains a variety of unique sequences, and also has improved amplification efficiency after panning.

### CROSS-REFERENCE TO RELATED APPLICATIONS

This application is a continuation of PCT Application No. PCT/KR2021/011388, filed on Aug. 25, 2021, which claims priority to Korean Patent Application Number 10-2020-0107932, filed on Aug. 26, 2020, both of which are hereby incorporated by reference in their entirety.

### REFERENCE TO AN ELECTRONIC SEQUENCE LISTING

The contents of the electronic sequence listing (OP20230013US.xml; Size: 90,174 bytes; and Date of Creation: Feb. 22, 2023) is herein incorporated by reference in its entirety.

### TECHNICAL FIELD

The present disclosure relates to a novel method of preparing an antibody library and the prepared library therefrom.

### BACKGROUND

A phage display technique is a technique by which bacteriophage using a bacterium as a host is genetically engineered to connect a genotype (gene) and a phenotype (protein) through a single phage particle. In this case, the gene as a genotype is inserted into a part of a phage gene, and the protein as a phenotype is displayed on a surface of the phage particle containing a gene of the protein. Such a physical combination of the genotype and phenotype, which is a very important concept in protein engineering, enables replication, amplification, analysis, and engineering by easily obtaining a gene of a protein clone selected by a property exhibited as a phenotype.

An antibody is an example to which, particularly, the phage display technique is very usefully applied. When an antibody library having very high diversity is displayed on a surface of a phage and allowed to bind to a surface-adsorbed antigen, a gene of an antibody clone selectively binding to the antigen can be obtained. This method is very effective in obtaining antibodies without using experimental animals, and has very high applicability in the development of therapeutic antibody drugs with less immune reaction in human bodies since, particularly, antibodies to certain antigens can be obtained. The quality of the library is important for obtaining good antibodies with high binding affinity, and particularly, the size and functional diversity of the library and the quality of clones are important.

The size of a library is one of the most important factors that determine the quality of antibodies selected from the library. An antigen-binding site of an antibody library has random diversity that is not theoretically biased toward any particular antigen, and an antibody selectively binding to a particular antigen by pure chance appears from the random diversity. Therefore, as the size of the library increases, i.e., as the number of different antibodies in the library increases, the likelihood of finding an antibody having high selectivity and affinity by chance is increased. Antibody libraries are generally considered to need a size of at least about 10⁸, and a large number of antibody libraries have a size of about 10⁹ to about 10¹¹.

The functional diversity of a library is the percentage of clones that can actually express antibodies among clones that constitute the library. Even though the size of a library is large, low functional diversity causes a decrease in substantial size of the library. The low functional diversity is largely due to errors in DNA synthesis and amplification during library construction. An antibody library is constructed through several polymerase chain reactions (PCRs), which inevitably results in a low frequency of errors due to the nature of enzymes and reactions, and the accumulation of such errors lowers the functional diversity of the final library. Particularly, in the case of a synthetic library, the possibility of introducing errors may be increased due to efficiency problems of base synthesis reactions. As described above, the problem of functional diversity tends to be more noticeable particularly in synthetic libraries, and most synthetic libraries need to be designed to avoid this problem.

The qualities of individual clones constituting a library, i.e., the expression level, stability, immunogenicity, and the like are factors that determine the performance of an antibody library. From an antibody engineering perspective, these factors need to be considered from designing a synthetic antibody library in order to select high-quality clones from the library. Particularly, when artificial diversity is introduced into existing antibody genes, the generated diversity needs to be designed to have compatibility with the antibody frameworks, and a radical change in the amino acid sequences of a natural antibody poses a risk of impeding the compatibility, stability and the like of an artificial synthetic antibody. Therefore, when artificial diversity is designed, efficient simulation of natural diversity is very important in the design and construction of a synthetic antibody library. Also, an antibody library composed of antibodies with various sequences may include sites where undesired protein modifications, such as glycosylation, oxidation, isomerization and deamidation, may occur, and these modifications may adversely affect the physical properties and industrial development of the antibodies.

When antibodies are produced in an animal body, antibody sequences having very high diversity are generated through the recombination of tens to hundreds of germline immunoglobulin genes present in the genome, and an antibody responding to a specific antigen is selected from among the antibody sequences. The binding strength of the selected antibody to the antigen is enhanced through hypermutation, finally resulting in a mature antibody. Therefore, the sequences of each mature antibody, particularly, the sequences of a complementarity determining region (CDR) that directly binds to an antigen is derived from germline gene sequences, while various sequences different from the germline CDR sequences are produced through recombination and mutation. In designing a synthetic antibody library, a construction strategy needs to be established that simulates CDR sequences of natural antibodies generated by such procedures.

Methods of producing antibodies from natural sources require considerable efforts and time for producing each antibody, and, thus, attempts to use synthetic antibody libraries have recently received attention. However, a conventional synthetic antibody library is constructed by randomly synthesizing variation sequences corresponding to CDRs, and, thus, the percentage of actually functioning antibodies is low or the efficiency of the library is not ensured. Also, Korean Patent Laid-open Publication No. 2016-0087766 discloses a problem that clones with low amplification efficiency were included in the library, which may degrade antibody production efficiency.

Accordingly, the present inventors have tried to build a synthetic human antibody library with high functional diversity and high quality as well as excellent amplification efficiency by panning to improve production efficiency. As a result, the present invention was completed by constructing a library using a method of predicting candidate sequences with excellent amplification efficiency by panning using a machine learning model.

### DISCLOSURE OF THE INVENTION

### Problems to be Solved by the Invention

The present disclosure provides a method of preparing an antibody library using a machine learning model and an antibody library prepared by the method.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides a method of preparing an antibody library, including individually designing complementarity determining region (CDR) sequences of antibodies; and synthesizing antibodies including the designed CDR sequences to prepare a library, when designing the CDR sequences individually, heavy chain complementarity determining region 3 (CDR-H3) is optimized using enrichment scores of candidate CDR-H3 sequences.

A second aspect of the present disclosure provides an antibody library prepared by the method of the first aspect.

### Effects of the Invention

The antibody library prepared by the preparation method according to an embodiment of the present disclosure includes antibodies having excellent physical properties to a large number of antigens, and thus can be favorably used as an antibody library that has functional diversity, includes a variety of unique sequences, and also has improved amplification efficiency after panning.

### BRIEF DESCRIPTION OF THE DRAWINGS

FIGS. 1A to 1H are diagrams showing frequencies of unique CDR sequences in designed and actual CDR repertoires of an OPALS library. The frequencies of appearance of each unique CDR sequence in the designed CDR repertoire and NGS-analyzed CDR repertoire of an actual scFv library are shown in XY-distribution plots. Each dot in the plots represents a unique CDR sequence. Only sequences in the designed repertoire were NGS analyzed, and CDR-H3 sequences were not analyzed since most of the sequences occur only once in the designed repertoire.

FIGS. 2A to 2C are diagrams showing variable domain sequence redundancies of the constructed OPALS library. The variable domain sequences of an unselected library were obtained through 300 bp paired-end sequencing on an Illumina MiSeq platform, and the number of replicates (n) for the variable domain sequences was analyzed. Approximately 98% of VH and Vλ sequences and approximately 88.5% of V_{K} sequences were found only once (n=1).

FIGS. 3A and 3B show the length distribution of designed CDRs and actual CDRs. In FIG. 3A, CDR-H2, L1 (kappa and lambda), and L3 (kappa and lambda) contain sequences with various lengths. Through a comparison between the length distribution in the designed repertoire and the length distribution in the next generation sequencing (NGS)-analyzed repertoire, the results was obtained indicating that shorter CDRs were preferred in the actual library. In FIG. 3B, the preference for the shorter CDRs was more evident in CDR-H3 having a wider range of length variation than other CDRs. In both FIG. 3A and FIG. 3B, the blue bars (“Designed”) indicate the frequency of each CDR length in the designed repertoire, and the orange bars (“Found”) indicate the frequency of each CDR length found from the NGS analysis of the constructed library.

FIG. 4 is a diagram showing the amino acid distribution of CDR-H3 in the OPALS library. The amino acid distribution of CDR-H3 of the natural human antibodies (N), the designed repertoire (D) and the actually constructed library (L) are shown for each position. Each stacked bar reflects the sum of frequencies of amino acids at each Kabat position of CDR-H3 with different lengths. For all CDR-H3s with different lengths, the last three residues are denoted by 100j, 101 and 102, respectively.

FIGS. 5A to 5H show changes in frequency in each CDR before and after protein A panning, depending on the origin of germline CDR sequences. The horizontal axis indicates the percentage frequency after protein A panning, and the vertical axis indicates the percentage frequency before panning.

FIG. 6 is a diagram showing a change pattern of amino acid frequency at each position of CDR-H3 after panning. The change pattern after panning of a total of 18 amino acids excluding Cys and Met at positions 95 to 98 of CDR-H3 having a length of 9 amino acids was analyzed. Fold >1 means an increase in amino acid after panning and fold <1 means a decrease in amino acid after panning (*: p<0.05, **: p<0.01).

FIGS. 7A to 7H are diagrams showing the prediction result of a machine learning model for each length of CDR-H3 in the validation set as the Area under the curve (AUC). The AUC representing the prediction results of the improvement of panning by the machine learning model is greater than 0.7 for all CDR-H3 lengths, meaning that the machine learning model of the present disclosure can predict the improvement of panning.

FIGS. 8A to 8H are diagrams showing the percentage of actual enriched sequences in all NGS-analyzed CDR-H3 sequences and machine learning-predicted CDR-H3 sequences. The percentage of sequences enriched by actual panning among sequences with predicted enrichment scores (ES)>0 after application of machine learning was significantly higher than the percentage of enriched sequences among all NGS-analyzed sequences before application of machine learning (p<0.0001).

FIG. 9 is a diagram showing the result of isolating PCR-amplified CDR-H3 by PAGE (polyacrylamide gel electrophoresis).

FIGS. 10A to 10C are diagrams schematically illustrating a method of preparing an improved antibody library (OPALT) of the present disclosure. In FIG. 10A, CDR-H3 oligonucleotides with different lengths were isolated by length by PAGE and used in the next phase. In FIG. 10B, CDR-H3 recovered by length by PAGE was amplified by PCR and combined with framework sequences to obtain a single CDR library. In-frame CDR sequences were enriched by panning of the library against protein A or L. In FIG. 10C, each CDR including contiguous framework regions was amplified and combined via a series of OE-PCRs to prepare an scFv library with six different CDRs.

FIGS. 11A and 11B are diagrams showing the results of dot blot assay of a single-CDR library before and after panning. FIG. 11A shows the percentages of solubly expressed in- frame clones before and after panning of a single CDR library, excluding CDR-H3. FIG. 11B shows the percentages of solubly expressed in-frame clones before and after panning of a single CDR library of CDR-H3 combined with a VHVL framework.

FIGS. 12A and 12B are diagrams showing the results of dot blot assay of eight sub-libraries of VH3VL1 and VH3VK3. Twenty-two clones randomly selected from each of the eight sub-libraries were analyzed by dot-blot, (FIG. 12A) VH3VL1^{˜#8} or (FIG. 12B) VH3VK3 #1^{˜}#8.

FIG. 13 is a diagram showing the frequency of CDR-H3 at different lengths in clones selected from OPALS and OPALT. Each bar indicates the frequency of the amino acid length of a CDR-H3 region in an antibody obtained by screening of each library. The frequencies of OPALS are clearly biased toward the shorter CDR-H3 (amino acids 9 and 10), whereas the frequencies of OPALT, particularly OPALT-λ, are more evenly distributed in length.

FIGS. 14A to 14N show the binding kinetics of target-specific scFv clones selected from OPALT and determined by SPR. Each SPR sensorgram shows binding of an scFv antibody derived from OPALT to an immobilized antigen. Kinetic rate constants for binding (kₐ), dissociation (k_{d}) as well as affinity (K_{D}) of the scFv antibody were measured. Of the 14 antibodies, CARS-B6 and CARS-D7 were obtained from OPALT-κ, and the remaining antibodies were obtained from OPALT-λ.

Concentration range of the antibody: 31.3 nM to 375 nM, CARS-B6; 54.5 nM to 872 nM, CARS-D7; 66.3 nM to 795 nM, CARS-D11; 5.7 nM to 91.4 nM, CARS-F4; BCMA-A3, 50 nM to 600 nM; 40 nM to 640 nM, BCMA-B5; BCMA-D11, 42.9 nM to 685 nM; 45 nM to 900 nM, CD22-D1; 40 nM to 480 nM, AIMP1-C6; 66.9 nM to 1070 nM, AIMP1-D4; 53.5 nM to 856 nM, AIMP1-E7; 57.1 nM to 914 nM, SerRS-D6; 79.5 nM to 233 nM, SerRS-F4.

The binding and dissociation rates were obtained by fitting the data to a simple Langmuir 1:1 binding model or a 1:1 drifting baseline model.

### BEST MODE FOR CARRYING OUT THE INVENTION

Hereinafter, embodiments and examples of the present disclosure will be described in detail with reference to the accompanying drawings so that the present disclosure may be readily implemented by those skilled in the art. However, it is to be noted that the present disclosure is not limited to the examples but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one part to another part includes both a case that a part is “directly connected or coupled to” another part and a case that a part is “indirectly connected or coupled to” another part via still another element such as another linker in the middle.

Through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

The term “phage display” used herein refers to the technique by which a gene of an external protein is fused to a gene of one of surface proteins of engineered genes of M13 bacteriophage and the external protein is fused to a surface protein of a produced phage to be displayed on a surface of the phage. When a protein is phage displayed, an external gene is often fused at the 5′ of the gIII gene.

The term “antibody” used herein refers to a protein specifically binding to a target antigen, and encompasses both of a polyclonal antibody and a monoclonal antibody. Also, the term encompasses any form produced by genetic engineering, such as chimeric antibodies (e.g., humanized murine antibodies) and heterogeneous antibodies (e.g., bispecific antibodies). Particularly, the antibody may be, but is not limited to, a heterotetramer consisting of two light chains and two heavy chains, and each of the chains may include a variable domain having a variable amino acid sequence and a constant domain having a constant amino acid sequence.

Throughout the present disclosure, the antibody includes IgA, IgD, IgE, IgM and IgG, and the subtypes of IgG include IgG1, IgG2, IgG3 and IgG4, and may include an antibody fragment. The term “antibody fragment” refers to a fragment having an antigen-binding function, and includes an Fc fragment, Fab, Fab′, F(ab′)2, scFv, a single variable domain antibody, Fv, and the like and also includes an antigen-binding form of the antibody. The term “Fc fragment” refers to an end region of an antibody, the end region being capable of binding to a cell surface receptor such as an Fc receptor, and is composed of second or third constant domains of two heavy chains of the antibody. Fab has a structure possessing variable regions of light chain and heavy chain, a constant region of light chain, and a first constant region (CH1) of heavy chain, and has one antigen-binding site. Fab′ is different from Fab in that the former has a hinge region including one or more cysteine residues at C-terminus of a heavy chain CH1 domain. F(ab′)2 antibody is formed through disulfide bonding of cysteine residues in the hinge region of Fab′. The term “Fv (variable fragment)” refers to a minimized antibody fragment having only a heavy chain variable region and a light chain variable region. The disulfide-stabilized variable fragment (dsFv) has a structure in which a heavy chain variable region and a light chain variable region are linked to each other through disulfide bonding, and the single chain variable fragment (scFV) generally has a structure in which a heavy chain variable region (VH) and a light chain variable region (VL) are covalently linked to each other by a peptide linker. The term “single variable domain antibody” refers to an antibody fragment composed of only one heavy or light chain variable domain.

Throughout the present disclosure, the antibody includes a recombinant single chain Fv fragment (scFv), and includes, without limitation, a bivalent or bispecific molecule, diabody, triabody and tetrabody.

Throughout the present disclosure, the term “Complementarity determining region, CDR)” used herein refers to a region that is three complementarity determining regions (CDRs) are present at each of light and heavy chain variable domains with particularly high variability of amino acid sequences, while antibodies specific to various antigens can be found due to the high variability. Three heavy chain CDRs sequentially from the amino terminus to the carboxyl terminus are called CDR-H1, CDR-H2 and CDR-H3, while three light chain CDRs sequentially from the amino terminus to the carboxyl terminus are called CDR-L1, CDR-L2 and CDR-L3. In one antibody, these six CDRs are assembled to form an antigen-binding site. Several methods of numbering amino acids in an antibody variable region sequence and defining the positions of CDRs are known. Particularly, the Kabat definition is used in the present disclosure.

The term “framework” used herein refers to a region other than the CDR in a variable domain sequence, and refers to a region which has lower sequence variability and diversity compared with the CDR and is not in general involved in an antigen-antibody reaction.

The term “immunoglobulin” used herein refers to a concept that encompasses an antibody and an antibody-like molecule having the same structural characteristics as an antibody and having no antigen specificity.

The term “germline immunoglobulin gene” used herein is an antibody gene that is present in animal germ cells and has not undergone the recombination of an immunoglobulin gene or the somatic hypermutation after differentiation into B cells. The number of germline immunoglobulin genes varies depending on the species of animal, but is generally tens to hundreds.

The term “mature antibody” used herein refers to an antibody protein which is expressed from an antibody gene prepared by the recombination of germline immunoglobulin genes or the somatic hypermutation through B cell differentiation.

The term “single chain Fv (scFv)” used herein refers to a protein in which light and heavy chain variable domains of an antibody are linked by a peptide chain linker of about 15 amino acids. The scFv protein may have an order of light chain variable domain-linker-heavy chain variable domain, or an order of heavy chain variable domain-linker-light chain variable domain, and may have the same or similar antigen specificity compared with its original antibody. The linker is a hydrophilic flexible peptide chain mainly composed of glycine and serine, and a sequence of 15 amino acids of “(Gly-Gly-Gly-Gly-Ser)3” or a similar sequence may be often used.

The term “antibody library” used herein refers to a collection of various antibody genes having different sequences. Very high diversity is required to isolate an antibody specific to any antigen from the antibody library, and in general, a library composed of 10⁹ to 10¹¹ different antibody clones is organized and used. The antibody genes constituting the antibody library are cloned to a phagemid vector and then transformed to E. coli.

The term “phagemid” vector used herein refers to a plasmid DNA having a phage origin of replication and usually has an antibiotic-resistant gene as a selection marker. The phagemid vector used in the phage display includes the gIII gene of the M13 phage or a part thereof, and a library gene is ligated to the 5′ end of the gIII gene and expressed as a fusion protein in E. coli.

The term “helper phage” used herein refers to a phage that provides necessary genetic information to allows packaging of the phagemid into a phage particle. Since only gIII of a phage gene or a part thereof exists in the phagemid, E. coli transformed by the phagemid is infected with the helper phage to supply the rest of phage genes. The types of helper phages include M13K07 or VCSM13, and most of the helper phages contain antibiotic-resistant genes, such as kanamycin, to allow the selection of E. coli infected with the helper phage. Also, the packaging signal is defective in the helper phage, and, thus, the phagemid gene, rather than the helper phage gene, is selectively packaged into a phage particle.

The term “panning” used herein refers to the process of selectively amplifying only those clones that bind to a specific molecule from a library of proteins, such as antibodies, displayed on a phage surface. The process is performed by adding a phage library to a target molecule immobilized on the surface to induce binding, washing and removing unbound phage clones, eluting only bound phage clones and infecting the E. Coli host again, and amplifying target-binding phage clones using helper phages. In most cases, this process is repeated three to four times or more to maximize the percentage of bound clones.

Hereinafter, embodiments and examples of the present disclosure will be described in detail with reference to the accompanying drawings. However, it is to be noted that the present disclosure is not limited to the embodiments, examples and drawings.

A first aspect of the present disclosure provides a method of preparing an antibody library, including individually designing complementarity determining region (CDR) sequences of antibodies; and synthesizing antibodies including the designed CDR sequences to prepare a library, when designing the CDR sequences individually, heavy chain complementarity determining region 3 (CDR-H3) is optimized using enrichment scores of candidate CDR-H3 sequences.

A human antibody library can be constructed by a method according to an embodiment of the present disclosure and a human antibody to any antigen can be obtained therefrom. Antibody libraries may be organized by a method of obtaining diversity from B cells contained in the bone marrow, spleen, blood, or the like, or by a method of obtaining diversity through artificial design and synthesis. The present disclosure provides the construction and validation of a synthetic human antibody library.

A phage-display antibody library prepared by the method according to an embodiment of the present disclosure is constructed in the form of a Fab or scFv fragment, which is a part of an immunoglobulin molecule. Since these fragments are smaller in size than 150 kDa immunoglobulin, the fragments can improve the efficiency of protein-engineered control and have the same antigen selectivity as immunoglobulin molecules. In the present disclosure, a library using an scFv fragment having a size of 25 kDa was constructed. Specifically, the library has a single polypeptide chain in which a VH domain and a VL domain of an immunoglobulin are linked by a chain consisting of 15 amino acids, i.e., (Gly-Gly-Gly-Gly-Ser)₃.

In an embodiment of the present disclosure, the design of the library sequence needs to be preceded in order to construct a synthetic library. Unlike B cell-derived antibody libraries or natural antibody libraries with relatively high framework diversity, the synthetic antibody library is constructed based on a single or limited number of framework sequences.

In an embodiment of the present disclosure, when the antibody library is constructed by the above-described method, two frameworks may be used. Specifically, the library was constructed such that all the clones constituting the library have, as frameworks, scFv having a human immunoglobulin IGHV3-23 gene and a human immunoglobulin IGKV3-20 gene linked by a linker, or scFv having a human immunoglobulin IGHV3-23 gene and a human immunoglobulin IGLV1-47 gene linked by a linker, and artificial diversity was introduced to complementarity determining regions of the frameworks. That is, various complementarity determining region (CDR) sequences were grafted into the frameworks of the library to construct an scFv antibody library.

In an embodiment of the present disclosure, the enrichment scores are predicted by a machine learning model, and the machine learning model may be trained by a) setting at least one CDR-H3 sequence(s) as input values, and b) setting, as result values, the enrichment scores calculated by measuring relative frequencies of the sequences before and after panning. More specifically, when candidate CDR-H3 sequences are input, the machine learning model may output enrichment scores which is predicted based on sequence information of the candidate CDR-H3 sequences (information such as amino acid length, amino acid composition ratio, amino acid residue type at each position, etc.).

In an embodiment of the present disclosure, the relative frequency of the sequences before and after panning may be measured by next-generation sequencing (NGS). Specifically, the relative frequency before and after panning may be measured by measuring the number of reads each representing a nucleic acid fragment analyzed by NGS.

The term “next generation sequencing (NGS)” used herein refers to the method of quickly decoding a vast amount of genomic information by decomposing the whole genome into countless fragments, reading each fragment in a massively parallel manner, and combining them by using computer technology. A large amount of sequencing data can be generated for a sample to be analyzed within a short time by NGS.

The term “machine learning” used herein refers to the ability of a computer to perform a necessary task without specific programming through continuous learning and prediction based on data without developing a pattern recognition task by a computer. Machine learning, a form of artificial intelligence, effectively automates a process of building an analytical model and allows a system to independently adapt to new scenarios. For example, the system can be trained to distinguish whether a received e-mail is spam or not through machine learning. The core of machine learning deals with representation and generalization. Representation refers to evaluation of data, and generalization refers to processing of unseen data. Specifically, machine learning focuses on prediction based on known properties learned from training data.

In an embodiment of the present disclosure, the machine learning model is trained to predict the enrichment scores for the candidate CDR-H3 sequences. Specifically, in order to train the machine learning model, sequence information of CDR-H3 including the enrichment scores for at least one CDR-H3 sequence(s) calculated based on NGS data (NGS read number, etc.) before and after panning and amino acid residue information for each position may be used as input values.

In an embodiment of the present disclosure, an enrichment score ESᵢ for a specific CDR-H3 sequence i may be calculated using following Equation 1.

$$Enrichment score ( ES ) = {log}_{2} \frac{{n}_{i - post} / {N}_{post}}{{n}_{i - pre} / {N}_{pre}} \times \sqrt{\frac{{n}_{i - post} + {n}_{i - pre}}{median ( {n}_{post} ) + median ( {n}_{pre} )}} \qquad [ Equation 1 ]$$

[Nₚᵣₑ: Total number of NGS reads of a library including at least one candidate CDR-H3 sequence(s) before panning, Nₚₒₛₜ: Total number of NGS reads of the library after panning, nᵢ₋ₚᵣₑ: Number of reads of a specific CDR-H3 sequence i in the library before panning, nᵢ₋ₚₒₛₜ: Number of reads of the specific CDR-H3 sequence i in the library after panning, nₚᵣₑ: Set of read numbers of individual CDR-H3 sequences in the library before panning, nₚₒₛₜ: Set of read numbers of individual CDR-H3 sequences in the library after panning, median(nₚᵣₑ): Median of the nₚᵣₑ, and median(nₚₒₛₜ): Median of the nₚₒₛₜ].

In an embodiment of the present disclosure, designing optimized sequences using the enrichment scores may be calculating or predicting the enrichment scores of candidate CDR-H3 sequences to select candidate CDR-H3 sequences with the enrichment scores calculated or predicted to be above 0. Also, predicting the enrichment score of the candidate sequence may be outputting a predicted enrichment score when the sequence information of the candidate sequence is input to the trained machine learning model, and when the predicted enrichment score is above 0, selecting the candidate sequence as a library sequence.

In an embodiment of the present disclosure, when designing the CDR sequences individually, in the case of CDR-H2, sequences derived from the VH1, VH4 or VH5 family may be excluded.

In an embodiment of the present disclosure, heavy chain complementarity determining region 1 (CDR-H1), heavy chain complementarity determining region 2 (CDR-H2), heavy chain complementarity determining region 3 (CDR-H3), light chain complementarity determining region 1 (CDR-L1), light chain complementarity determining region 2 (CDR-L2) and light chain complementarity determining region 3 (CDR-L3), which constitute the complementarity determining regions of the antibodies included in the antibody library, may have polymorphism, and a method of designing/constructing CDR-H1, CDR-H2, CDR-L1, CDR-L2 and/or CDR-L3 among the CDRs may employ an antibody library preparation method known in the art.

In an embodiment of the present disclosure, when designing the CDR sequences individually, for CDR-H1, CDR-H2, CDR-L1 or CDR-L2, the sequences therefor may be designed by simulating i) an utilization frequency of each germline immunoglobulin gene, ii) a frequency of mutation into each of 20 amino acids by somatic hypermutations at each amino acid position, iii) a frequency of each CDR sequence length or iv) a frequency of each amino acid at each position calculated by analyzing a combination thereof, of CDRs of actual human-derived mature antibodies.

In an embodiment of the present disclosure, when designing the CDR sequences individually, for CDR-L3,a) 7 to 8 amino acid sequences from an N-terminus of the CDR may be designed by simulating i) an utilization frequency of each germline immunoglobulin gene, ii) a frequency of mutation into each of 20 amino acids by somatic hypermutations at each amino acid position, iii) a frequency of each CDR sequence length or iv) a frequency of each amino acid at each position calculated by analyzing a combination thereof, of CDRs of actual human-derived mature antibodies, and b) 2 to 3 amino acid sequences from a C-terminus of the CDR are designed by analyzing and calculating a frequency of each amino acid at each position in the CDRs of actual human-derived mature antibodies, then simulating sequences that reflect the calculated frequencies; and the CDR-L3 contains 9 to 11 amino acids, the analysis of the frequencies is conducted according to each length, and the CDR-L3 sequences being designed based on an analysis result of complementarity determining region CDR-L3 of human-derived mature antibodies, which have the same amino acid lengths as CDR-L3 to be designed.

In an embodiment of the present disclosure, when designing the CDR sequences individually, for CDR-H3, a) each sequence therefor excluding 3 amino acids from a C-terminus of the CDR may be designed by reflecting a frequency of each amino acid at each position of CDRs of actual human-derived mature antibodies, and b) 3 amino acid sequences from the C-terminus of the CDR are designed by analyzing and calculating frequencies of the 3 amino acid sequences at the corresponding positions of CDRs of actual human-derived mature antibodies, then simulating sequences that reflect the calculated frequencies; and the CDR-H3 contains 9 to 16 amino acids, the analysis of the frequencies is conducted according to each length, and the CDR-H3 sequences being designed based on an analysis result of complementarity determining region CDR-H3 of human-derived mature antibodies, which have the same amino acid length as CDR-H3 to be designed.

In an embodiment of the present disclosure, the frequency of use of each of the germline CDR sequences may simulate the frequency of use of each germline CDR sequence in natural human antibodies, which is obtained by analysis of antibody sequence databases.

In an embodiment of the present disclosure, designing CDR-H3 of the method may be selecting optimized sequences using the machine learning model, after designing sequences simulated by the above-described method, or designing an antibody library by the preparation method known in the art.

In an embodiment of the present disclosure, in the case of designing light chain in the CDR sequences, the light chain may be a kappa light chain or lambda light chain. Specifically, in designing light chain CDR, a CDR can be designed for each of a kappa light chain and lambda light chain, and when designing each light chain variable region for the kappa light chain and the lambda light chain, the kappa light chain CDR may be assembled by linkage with a kappa light chain framework of IGKV3-20 and the lambda light chain CDR may be assembled by linkage with a lambda light chain framework of IGLV1-47.

The term “simulation” used herein refers to the designing of sequences by reflecting the expression frequency or modification frequency of amino acid sequences or the like to perform random simulation, and encompasses the meaning of simulating the expression frequency or modification frequency of amino acid sequences of a natural antibody, particularly a human-derived mature antibody.

In an embodiment of the present disclosure, the method may further include, after the designing of the complementarity determining region amino acid sequences, excluding sequences with the possibility of N-glycosylation, isomerization, deamidation, cleavage and oxidation from the designed sequences.

In an embodiment of the present disclosure, when designing CDR-H3 sequences in the above-described method, isolating and recovering CDR-H3 sequences with different lengths by length may be further included. Specifically, CDR-H3 sequences composed of 9 to 16 amino acids may be isolated by length of each amino acid.

In an embodiment of the present disclosure, the method may further include, after the designing of the complementarity determining region amino acid sequences, reverse-translating the designed sequences into polynucleotide sequences and then designing oligonucleotide sequences in which framework region sequences of human antibody germline variable domain genes are linked to the 5′ and 3′ ends of the reverse-translated polynucleotides.

In an embodiment of the present disclosure, the antibodies may include amino acid sequences encoded by IGHV3-23 (VH3-23, Genebank accession No. Z12347), IGKV3-20 (VK3-A27, Genebank accession No. X93639), IGLV1-47 (VL1g, GenBank accession No. Z73663) or fragments thereof, specifically, the antibodies may include amino acid sequences encoded by IGHV3-23, IGKV3-20, IGLV1-47 or fragments thereof as frameworks for constructing an antibody library.

In an embodiment of the present disclosure, the framework sequence for constructing the antibody library in the above-described method may be an amino acid sequence of SEQ ID NO: 70 or SEQ ID NO: 71.

In an embodiment of the present disclosure, the antibodies may be selected from the group consisting of IgA, IgD, IgE, IgM, IgG, Fc fragments, Fab, Fab′, F(ab′)₂, scFv, single variable domain antibody and Fv.

In an embodiment of the present disclosure, the method may further include deimmunizing the designed CDR sequences. Specifically, the deimmunization may be performed to exclude potential immunogenic sequences by excluding sequences predicted to have a strong bond to at least one of MHC class II allele(s) at an in silico level. Also, the MHC class II allele is an HLA-DRB gene and may be at least one selected from the group consisting of DRB1*01:01, DRB1*03:01, DRB1*03:02, DRB1*04:01, DRB1*04:04, DRB1*04:05, DRB1*07:01, DRB1*08:02, DRB1*08:03, DRB1*09:01, DRB1*11:01, DRB1*13:01, DRB1*13:02, DRB1*12:02, DRB1*14:01, DRB1*15:01, DRB1*15:03, DRB3*01:01, DRB4*01:01 and DRB5*01:01.

A second aspect of the present disclosure provides an antibody library prepared by the method of the first aspect. The contents overlapping with those of the first aspect are also applied to the library of the second aspect.

In an embodiment of the present disclosure, the antibodies may include amino acid sequences encoded by IGHV3-23 (VH3-23, Genebank accession No. Z12347), IGKV3-20 (VK3-A27, Genebank accession No. X93639), IGLV1-47 (VL1g, GenBank accession No. Z73663) or fragments thereof, specifically, the antibodies may include amino acid sequences encoded by IGHV3-23, IGKV3-20, IGLV1-47 or fragments thereof as frameworks for constructing an antibody library.

In an embodiment of the present disclosure, the antibody may include an amino acid sequence of SEQ ID NO: 72 or SEQ ID NO: 74.

In an embodiment of the present disclosure, the CDR-H3 sequences of the antibody may be sequences optimized for panning efficiency by the machine learning model.

In an embodiment of the present disclosure, the CDR-H2 sequences of the antibody may exclude sequences derived from the VH1, VH4 or VH5 family.

Hereinafter, the present disclosure will be explained in more detail with reference to Examples. However, the following Examples are illustrative only for better understanding of the present disclosure but do not limit the present disclosure.

### MODE FOR CARRYING OUT THE INVENTION

### EXAMPLES

### Example 1: Next Generation Sequencing Analysis

After three rounds of panning a synthetic scFv antibody library [PLoS One. 2015;10: 1-18, Bai et al, Korean Patent Laid-open Publication No. 2016-0087766] with over 8,000 individually designed sequences for each complementarity-determining region (CDR) against protein A, sequencing was performed using an Illumina Miseq platform with ^{˜}350 bp paired-end reads. For ease of analysis of 1.5 to 2.3 million sequences obtained by NGS for each variable domain (VH, VL and VK), they were divided into FASTA files containing ^{˜}10⁵ sequences. The CDR sequences were extracted from the FASTA files by using a self-developed Python script in .csv(comma-separated values) format. The germline origins of individual CDR-H1, -H2, -L1, -L2 and -L3 sequences were determined by using a self-developed VBA program. As for CDR-H3, the extracted sequences were sorted by length (9 to 20 amino acids).

### Example 2: Design of CDR Sequences

CDR sequences except for CDR-H3 were designed according to previously reported literature [PLoS One. 2015;10: 1-18, Korean Patent Laid-open Publication No. 2016-0087766]. Then, the designed sequences were evaluated through potential bonding to human MHC class 2 molecules by using the netMHCIIpan-3.1 software. Alleles used for evaluation were DRB1*01:01, DRB1*03:01, DRB1*03:02, DRB1*04:01, DRB1*04:04, DRB1*04:05, DRB1*07:01, DRB1*08:02, DRB1*08:03, DRB1*09:01, DRB1*11:01, DRB1*13:01, DRB1*13:02, DRB1*12:02, DRB1*14:01, DRB1*15:01, DRB1*15:03, DRB3*01:01, DRB4*01:01, and DRBS*01:01, and the combinations thereof account for 81.2%, 75.1%, 71.3% and 61.7% of the Caucasian, Korean, Black and Hispanic populations, respectively (http://allelefrequencies.net). Then, 9-amino acid (aa) fragment sequences overlapping with each other in a fragment sequence in which each CDR sequence and 8 amino acid sequences of framework regions adjacent to both sides thereof were added were analyzed, and CDR sequences expected to have strong bonding to MHC class 2 (with top 0.5% binding strength among random 9-aa amino acid sequences) was excluded from a library design.

As for CDR-H3, the relative frequencies of each individual CDR sequence (a total of 7,526 unique sequences) were compared before and after three rounds of panning against protein A, which is known to bind to the VH3 family heavy chain variable region of a human antibody, and an enrichment score ESᵢ for any specific CDR-H3 sequence i in the antibody library including these sequences is represented by following equation:

$$Enrichment score ( ES ) = {log}_{2} \frac{{n}_{i - post} / {N}_{post}}{{n}_{i - pre} / {N}_{pre}} \times \sqrt{\frac{{n}_{i - post} + {n}_{i - pre}}{median ( {n}_{post} ) + median ( {n}_{pre} )}} \qquad [ Equation 1 ]$$

Herein, Nₚᵣₑ and Nₚₒₛₜ are the total numbers of CDR-H3 reads through NGS analysis of the antibody library before and after panning, respectively, nᵢ₋ₚᵣₑ and nᵢ₋ₚₒₛₜ are the numbers of reads of the specific CDR-H3 sequence i in the antibody library before and after panning, respectively, nₚᵣₑ and nₚₒₛₜ are sets of read numbers of individual CDR-H3 sequences in the antibody library before and after panning, respectively, and median(nₚᵣₑ) and median(nₚₒₛₜ) are medians of read numbers of the specific CDR-H3 sequence before and after panning, respectively.

The CDR-H3 sequences and their ESs were used to train a machine learning model for efficient prediction of enriched sequences. Herein, 70% of the CDR-H3 sequence-ES data was used as a training set and the remaining 30% was used as a validation set. Amazon Machine Learning (https://console.aws.amazon.com/machinelearning/home, 2017) was used to construct and evaluate a machine learning model for prediction of enriched sequences. To train the machine learning model, a .csv (comma-separated values) file containing the CDR sequences for each CDR-H3 length, their ES, and amino acid residues at each position in each sequence was generated using the following parameters: maximum machine learning model size, 100 MB; maximum number of data passes, 100; L2 norm, mild (10⁻⁶). Consequently, the machine learning model was validated by using the validation set.

Preliminary CDR-H3 sequence repertoires with different lengths were designed according to previously reported literature [PLoS One. 2015;10: 1-18, Korean Patent Laid-open Publication No. 2016-0087766]. The designed sequences were evaluated by the above-described machine learning model for prediction of enrichment by phage display, and sequences with predicted ES>0 (i.e., relative numbers increased by panning) were selected for construction of a library. The selected sequences were further analyzed for HLA-DRB binding as described above to identify and remove potential immunogenic sequences. The designed CDR sequences with adjacent framework sequences and adapter sequences for PCR amplification were reverse-translated into DNA sequences and synthesized into an oligo pool (LC Sciences, Houston, TX, USA).

### Example 3: Isolation of CDR-H3 by PAGE (Polyacrylamide Gel Electrophoresis)

The oligo pool synthesized as described above was amplified by PCR using primers annealing to the adapter sequences (see Tables 2 and 8 for information on the primers and primer sequences used for PCR). From the amplified oligo pool, each CDR repertoire was amplified using framework-specific primers and purified by agarose gel electrophoresis. CDR-H3 oligonucleotides amplified by PCR in a formamide loading buffer [80% formamide, 1 mg/mL xylene cyanol, 1 mg/mL bromophenol blue, and 10 mM EDTA; pH 8.0)] were isolated by their length on a 10% denaturing polyacrylamide gel [10% acrylamide:bisacrylamide [19:1], 8 M urea, 0.1 volume of 10× Tris-borate-EDTA [TBE] buffer; pH 8.0]. DNA bands were visualized by SYBR gold staining (Thermo Fisher), cleaved with a clean scalpel, and transferred to a microfuge tube. Then, 2 volumes of elution buffer [0.5 M ammonium acetate, 15 mM magnesium acetate, 1 mM EDTA, and 0.1% SDS)] were added to gel slices and incubated at 37° C. After centrifugation, the supernatant was transferred to a new microfuge tube and 0.5 volumes of elution buffer was added to polyacrylamide pellets and briefly vortexed. The mixture was centrifuged again and the supernatant was combined with the previous supernatant and filtered using a Spin-X centrifuge tube filter (Corning 8160, Corning, USA). Then, 2 volumes of ethanol was added to the filtrate and left to stand on ice for 30 minutes. The precipitated DNAs were pelleted by centrifugation and redissolved in 200 μL of 1× Tris-EDTA (TE) buffer (pH 7.6), followed by addition of 25 μL of 3 M sodium acetate (pH 5.2) and 550 μL of ethanol to re-precipitate the DNAs. After standing on ice for 30 minutes and centrifugation, the precipitated DNA pellets were washed with cold 70% ethanol and dissolved in 10 μL of 1× TE (pH 7.6). reaction)

### Example 4: Amplification of Oligonucleotide by PCR (Polymerase Chain Reaction)

An array-synthesized CDR oligonucleotide mixture (Oligomix™, LC Sciences, TX, Houston, USA) was dissolved in 25 μL of nuclease-free water. The dissolved oligonucleotide pool and the PAGE-purified CDR-H3 oligonucleotides were amplified by PCR with specific primer sets (Table 1; primer sequences shown in Table 7). PCR was performed in a volume of 100 μL while a template DNA (CDR oligonucleotide mixture; CDR-H3 isolated by amino acid length and named H3 #1 to #8), forward and reverse amplification primers at a final concentration of 0.6 μM each, 10 μL of Taq polymerase buffer (NEB), 0.2 mM each dNTP (NEB; New England Biolabs) and 2.5 units of Taq DNA polymerase (NEB) were added. A PCR thermal cycle was performed under the following conditions: initial fusion at 94° C. for 5 min, followed by 30 cycles at 94° C. for 30 sec, 56° C. for 30 sec and 72° C. for 30 sec, and a final extension at 72° C. for 7 min. The PCR product was electrophoresed on a 2% agarose gel and bands were identified under UV light. According to the manufacturer's protocol, a gel band with a length close to 100 bp was cleaved and DNAs were extracted from the agarose gel slice by using a DNA gel extraction kit (QIAGEN; QIAquick Gel Extraction Kit).

TABLE 1
Primer set information for amplification
of CDR from oligonucleotide mixture

| | | |
|---|---|---|
| Product name | Template | Primer |

| | | | |
|---|---|---|---|
| CDR-H1 | Oligomix | VH3-1-f | VH3-1-b |
| CDR-H2 | Oligomix | VH3-2-f | VH3-2-b |
| Oligo-H3 | Oligomix | lib-cdr-f | lib-cdr-b |
| CDR-H3(#1~#8) | H3(#1~#8) | VH-3-f | VH-3-b |
| CDR-L1 | Oligomix | VL-1-f | VL-1-b |
| CDR-L2 | Oligomix | VL-2-f | VL-2-b |
| CDR-L3 | Oligomix | VL-3-f | VL-3-b |
| CDR-K1 | Oligomix | VK-1-f | VK-1-b |
| CDR-K2 | Oligomix | VK-2-f | VK-2-b |
| CDR-K3 | Oligomix | VK-3-f | VK-3-b |

### Example 5: Construction of Single-CDR scFv Library

Human germline immunoglobulin variable segments IGHV3-23, IGLV1-47 and IGKV3-20were synthesized and cloned into the (Genscript, Piscataway, NJ, USA) pUC57 vector to be used as a framework for library construction. Also, the framework gene was cloned into pFcF (a pcDNA3.1-based vector with human IgG1 Fc and a pair of asymmetric Sfil sites) to perform PCR amplification by using a different primer set (Table 2) to suppress cross-amplification. As a result, the scFv framework gene (VHVL) composed of IGHV3-23/IGLV1-47 and the scFv framework gene (VHVK) composed of IGHV3-23/IGKV3-20 were cloned into pUC57 and pFcF, respectively, and used as PCR templates. A PCR mixture (100 μL) for amplification of the framework was prepared as follows: 200 ng of template DNA, forward and reverse amplification primers at a final concentration of 0.6 μM each, 0.2 mM each dNTP, 10 μL of Taq polymerase buffer, 2.5 units of Taq DNA polymerase and nuclease-free water. A PCR amplification thermal cycle was performed under the following conditions: initial fusion at 94° C. for 5 min, followed by 30 cycles at 94° C. for 30 sec, 56° C. for 30 sec and 72° C. for 30 sec, and a final extension at 72° C. for 7 min. The framework PCR product amplified by the above method was purified by 1% agarose gel electrophoresis.

The amplified CDRs were grafted to template scFv sequences by overlap extension PCR (Table 3) in a volume of 100 μL with 0.6 μM of amplification primers (pUC57-in-b and hCH2-in-b), 10 μL of Taq polymerase buffer, 0.2 mM each dNTP and 2.5 units of Taq DNA polymerase. An amplification thermal cycle was performed under the following conditions: initial fusion at 94° C. for 5 min, followed by 30 cycles at 94° C. for 30 sec, 56° C. for 30 sec and 72° C. for 70 sec, and a final extension at 72° C. for 7 min. The DNA amplified by the above method was purified by 1% agarose gel electrophoresis.

The PCR product and the pComb3× vector were cleaved with a restriction enzyme Sfil (Roche) overnight at 50° C. and purified by 1% agarose gel electrophoresis as described above. An scFv DNA (^{˜}750 bp) cleaved with the Sfil was ligated to a pComb3× phagemid vector under the following conditions: 1μg of insert, 1.5 μg of vector DNA, 10 μL of T4 ligase buffer(10×) and 1,600 units of T4 DNA ligase (New England Biolabs) and 100 μL of final reactant. The ligation mixture reacted overnight at room temperature was added with 10 μL of 3 M sodium acetate (pH 5.2) and 256 μL of ethanol, followed by standing at -20° C. for 2 hours to precipitate the ligated DNA. The precipitated DNAs were pelleted by centrifugation at 14,000×g and washed twice with cold 70% ethanol. The DNA pellets were air-dried and dissolved in 10 μL of 10% glycerol. Then, for electroporation, the ligated DNA was mixed with 50 μL of electrocompetent TG1 cells (Lucigen, Middleton, WI, USA) and added to an electroporated cuvette (1 mm spacing; Bio-Rad, Hercules, CA, USA). The cuvette was left to stand on ice for 1 minute and a single 2.50 kV pulse was applied thereto using a MicroPulser electroporator (Bio-Rad). After electroporation, 1 mL of warm recovery medium (Lucigen) was immediately added to the cuvette to resuspend the cells. The recovery was repeated once more with 1 mL of fresh recovery medium, and the bacterial suspensions were combined. Transformed cells were incubated at 37° C. for 1 hour with stirring at 250 rpm. To estimate transformation titers, cells were diluted to 10⁻³ and 10⁻⁴ and plated on LB-ampicillin agar plates. The remaining cells were centrifuged at 3,500×g, resuspended in 200 μL of LB medium, plated on 150 mm LB-ampicillin agar plates supplemented with 2% (w/v) glucose, and incubated overnight at 37° C. On the next day, 5 mL of SB medium (3% tryptone, 2% yeast extract, 1% MOPS, pH 7.0) was added to the plates and the bacteria were scraped off with a flame-sterilized glass spreader. Then, 0.5 volumes of 50% glycerol were added to the resuspended E. coli and mixed thoroughly. Thereafter, 1 mL of aliquot was quick-frozen in liquid nitrogen and stored at −80° C.

TABLE 2
Primer information for amplification of framework
region for single-CDR library construction

| | | |
|---|---|---|
| Product name | Template | Primer |

| | | | |
|---|---|---|---|
| 1 | VHVL-pUC57 | pUC57-b | VH3-1-f-rc |
| 2 | VHVL -pUC57 | pUC57-b | VH3-2-f-rc |
| 3 | VHVL -pUC57 | pUC57-b | VH-3-rc-F |
| 4 | VHVL -pUC57 | pUC57-b | VL-1-f-rc |
| 5 | VHVL -pUC57 | pUC57-b | VL-2-f-rc |
| 6 | VHVL -pUC57 | pUC57-b | VL-3-f-rc |
| 7 | VHVK -pUC57 | pUC57-b | VK-1-f-rc |
| 8 | VHVK -pUC57 | pUC57-b | VK-2-f-rc |
| 9 | VHVK -pUC57 | pUC57-b | VK-3-f-rc |
| 10 | VHVL -pFcF | VH3-1-b-rc | hCH2-b |
| 11 | VHVL - pFcF | VH3-2-b-rc | hCH2-b |
| 12 | VHVL - pFcF | VH-3-rc-B | hCH2-b |
| 13 | VHVL - pFcF | VL-1-b-rc | hCH2-b |
| 14 | VHVL - pFcF | VL-2-b-rc | hCH2-b |
| 15 | VHVL - pFcF | VL-3-b-rc | hCH2-b |
| 16 | VHVK - pFcF | VK-1-b-rc | hCH2-b |
| 17 | VHVK - pFcF | VK-2-b-rc | hCH2-b |
| 18 | VHVK - pFcF | VK-3-b-rc | hCH2-b |

TABLE 3
Assembly of single-CDR scFv libraries by overlap extension PCR

| Product name | Template | Primer | |
|---|---|---|---|
| VHVL -H1 | 1 + CDR-H1 + 10 | pUC57-in-b | hCH2-in-b |
| VHVL -H2 | 2 + CDR-H2 + 11 | | |
| VHVL -H3 | 3 + CDR-H3 + 12 | | |
| VHVL -L1 | 4 + CDR-L1 + 13 | | |
| VHVL -L2 | 5 + CDR-L2 + 14 | | |
| VHVL -L3 | 6 + CDR-L3 + 15 | | |
| VHVK -K1 | 7 + CDR-K1 + 16 | | |
| VHVK -K2 | 8 + CDR-K2 + 17 | | |
| VHVK -K3 | 9 + CDR-K3 + 18 | | |

### Example 6: Proofreading of Synthetic CDR

A single-CDR scFv phage library was proofread by panning once or twice against protein A or protein L immobilized on the surface. Specifically, 1 μg/mL protein A or L in 1× PBS was immobilized in an immunotube for 1 hour, and the immunotube was blocked with 1× PBS(mPBS) containing 3% nonfat dry milk. After blocking at room temperature for 1 hour, the rescued single CDR library (10¹⁰ cfu) in 1 mL of mPBS was added to the immunotube and incubated at 37° C. for 2 hours, and the immunotube was washed 3 times with PBST. The bound phages were eluted with 1 mL of 100 mM triethylamine and neutralized by addition of 0.5 mL of 1 M Tris (pH 7.4). The neutralized phages were added to 8.5 mL of mid-log phase TG1 E. coli cells and incubated at 37° C. for 1 hour with gentle stirring. TG1 E. coli (OP600=0.7) infected with the phages was incubated overnight on LB plates containing 100 μg/mL ampicillin (LB-amp plate) and 2% glucose. On the next day, the incubated bacteria were harvested from the plates. The experimental conditions were slightly different for each CDR, and specific experimental conditions are described in Table 4.

TABLE 4
Panning conditions for single-CDR library

| | VH-H1, VH- | | | | | | | | | | VH-H3 #1~#8 | |
|---|---|---|---|---|---|---|---|---|---|---|---|---|
| | H3 #1~#8 (λ) | | VH-H2 | | L1 | | L2, L3 | K1, K3 | K2 | | (κ) | |
| Panning on: | Protein A | | Protein A | | Protein A | | Protein A | Protein L | Protein L | | Protein L | |
| # of rounds | 1 | | 2 | | 2 | | 1 | 1 | 1 | | 1 | |
| Washing | 10 | | 10 | | 10 | | 3 | 3 | 10 | | 10 | |
| cycles | | | | | | | | | | | | |
| Tween20 | X | | X | | X | | ◯ | ◯ | X | | X | |
| Glucose | X | | X | | ◯ | | X | X | ◯ | | ◯ | |
| Rescue time | 5 | h | 5 | h | 4 | h | O/N* | O/N* | 5 | h | 4 | h |
| Infection | 15 | min | 15 | min | 15 | min | 1 h | 1 h | 15 | min | 15 | min |

time

*O/N: overnight incubation

### Example 7: Construction of Final scFv Library

500 μL of aliquot of each single-CDR library proofread according to Example 6 was centrifuged at 13,000 rpm for 15 minutes. The supernatant was discarded, the plasmid DNA was extracted from the E. coli pellets with a miniprep kit (QIAGEN), and the proofread CDRs and adjacent framework sequences were amplified by PCR (Table 5). Amplification thermal cycle conditions were as follows: initial fusion at 94° C. for 5 min, followed by 25 cycles at 94° C. for 30 sec, 56° C. for 30 sec and 72° C. for 30 sec, and a final extension at 72° C. for 7 min. Assembly of V_{H} and V_{L}/V_{K} fragments was performed by OE-PCR (Table 6): initial fusion at 94° C. for 5 min, followed by 30 cycles at 94° C. for 30 sec, 56° C. for 30 sec and 72° C. for 1 min, and a final extension at 72° C. for 7 min. The amplified variable domains were assembled into the final scFv library by OE-PCR (Table 6): initial fusion at 94° C. for 5 min, followed by 30 cycles at 94° C. for 30 sec, 56° C. for 30 sec and 72° C. for 1.5 min, and a final extension at 72° C. for 7 min. Four 100 μL reactions were performed in parallel, the products were combined, and then DNAs were precipitated as described above. The precipitated DNAs were dissolved in 50 μL of nuclease-free water and purified using a 1% agarose gel band (^{˜}1,200 bp) and a DNA gel extraction kit. The purified product was cleaved with a restriction enzyme Sfil and ligated into a pComb3× vector cleaved with Sfil. The ligated DNA was transformed to TG1 electrocompetent E. coli as described above. The transformed bacteria were grown overnight on square plates containing 100 μg/mL ampicillin (LB-amp plate) and 2% (w/v) glucose. On the next day, 10 mL of SB medium was added to the square plates(245×245×20 mm, SPL), the bacteria were harvested from the square plates, and pellets obtained by centrifugation were resuspended in 2 mL of SB medium. Glycerol was added to a final concentration of ^{˜}15% (0.5 volumes of 50% glycerol) and mixed thoroughly, and 1 mL of aliquot was quick-frozen in liquid nitrogen and stored at −80° C.

TABLE 5
Amplification of proofread CDR and
adjacent framework region by PCR

| | | |
|---|---|---|
| Product | Template | Primer |

| | | | |
|---|---|---|---|
| VH3-1 | VHVL -H1 | pC3x-f | VH3-1-b |
| VH3-2 | VHVL -H2 | VH3-1-b-rc | VH3-2-b |
| VH3-H3 | VHVL -H3 | VH3-2-b-rc | VH-3-b |
| VL-1 | VHVL -L1 | VH-3-rc-B | VL-2-f-rc |
| VL-2 | VHVL -L2 | VL-2-f | VL-3-f-rc |
| VL-3 | VHVL -L3 | VL-3-f | pC3x-b |
| VK-1 | VHVK -K1 | VH-3-rc-B | VK-2-f-rc |
| VK-2 | VHVK -K2 | VK-2-f | VK-3-f-rc |
| VK-3 | VHVK -K3 | VK-3-f | pC3x-b |

TABLE 6
Primers for assembly of VH, VL/VK and scFv by OE-PCR

| | | |
|---|---|---|
| Product | Template | Primer |

Primer for assembly of VH or VL/VK fragment

| | | | |
|---|---|---|---|
| VH3 | VH3-(1 + 2) + VH3-H3 | pC3x-f | VH-3-b |
| VL1 | VL-(1 + 2 + 3) | VH-3-rc-B | pC3x-b |
| VK3 | VK-(1 + 2 + 3) | VH-3-rc-B | pC3x-b |

Amplification of fianl scFv gene

| | | | |
|---|---|---|---|
| VH3VL1 | VH3 + VL1 | pC3x-f | pC3x-b |
| VH3VK3 | VH3 + VK3 | pC3x-f | pC3x-b |

TABLE 7
Primer sequences used in the present disclosure

| | | | |
|---|---|---|---|
| Product | Primer name | Sequence(5′-3′) | Sequence No. |

Primer for producing CDR

| | | | |
|---|---|---|---|
| VH3 CDR-H1 | VH3-1-f | CTCCGGATTCACTTTCAGC | 1 |
| | VH3-1-b | TTACCTGGTGCCTGTCTG | 2 |
| VH3 CDR-H2 | VH3-2-f | GGACTGGAGTGGGTCTCT | 3 |
| | VH3-2-b | GCGTGAGATGGTGAAGCG | 4 |
| Oligo-H3 | lib-cdr-f | GTCAGTCACGCTCTAAGG | 5 |
| | lib-cdr-b | CTGAGTCGATGACCTACG | 6 |
| CDR-H3 | VH-3-f | CACTGCCGTGTATTACTGC | 7 |
| | VH-3-b | CAGAGTACCTTGTCCCCA | 8 |
| CDR-L1 | VL-1-f | CGCGTCACCATCAGCTGC | 9 |
| | VL-1-b | CTGGGAGTTGCTGATACCA | 10 |
| CDR-L2 | VL-2-f | CTCCTAAGCTCCTGATTTAC | 11 |
| | VL-2-b | AAAGCGATCAGGCACACC | 12 |
| CDR-L3 | VL-3-f | CGAGGCTGACTATTACTGC | 13 |
| | VL-3-b | AGTTTGGTCCCACCGCCG | 14 |
| CDR-K1 | VK-1-f | CGCGCAACTCTGTCTTGT | 15 |
| | VK-1-b | CCAGGTTTCTGTTGGTACCA | 16 |
| CDR-K2 | VK-2-f | CCACGCCTGCTCATCTAT | 17 |
| | VK-2-b | GAACCTGTCTGGGATGCC | 18 |
| CDR-K3 | VK-3-f | GACTTCGCAGTTTACTATTGT | 19 |
| | VK-3-b | ACCTTCGTTCCCTGACCA | 20 |

Primer for construction of framework

| | | | |
|---|---|---|---|
| 5′ fragment- | pUC57-b | TTCGCCATTCAGGCTGCG | 21 |
| common | | | |
| 5′ fragment-pUC57 | VH3-1-f-rc | GCTGAAAGTGAATCCGGAG | 22 |
| | VH3-2-f-rc | AGAGACCCACTCCAGTCC | 23 |
| | VH-3-rc-F | GCAGTAATACACGGCAGTG | 24 |
| | VL-1-f-rc | GCAGCTGATGGTGACGCG | 25 |
| | VL-2-f-rc | GTAAATCAGGAGCTTAGGAG | 26 |
| | VL-3-f-rc | GCAGTAATAGTCAGCCTCG | 27 |
| | VK-1-f-rc | ACAAGACAGAGTTGCGCG | 28 |
| | VK-2-f-rc | ATAGATGAGCAGGCGTGG | 29 |
| | VK-3-f-rc | ACAATAGTAAACTGCGAAGTC | 30 |
| 3′ fragment- | hCH2-b | CTTGACCTCAGGGTCTTC | 31 |
| common | | | |
| 3′ fragment-pFcF | VH3-1-b-rc | CAGACAGGCACCAGGTAA | 32 |
| | VH3-2-b-rc | CGCTTCACCATCTCACGC | 33 |
| | VH-3-rc-B | TGGGGACAAGGTACTCTG | 34 |
| | VL-1-b-rc | TGGTATCAGCAACTCCCAG | 35 |
| | VL-2-b-rc | GGTGTGCCTGATCGCTTT | 36 |
| | VL-3-b-rc | CGGCGGTGGGACCAAACT | 37 |
| | VK-1-b-rc | TGGTACCAACAGAAACCTGG | 38 |
| | VK-2-b-rc | GGCATCCCAGACAGGTTC | 39 |
| | VK-3-b-rc | TGGTCAGGGAACGAAGGT | 40 |

Primer for amplification of single-CDR library

| | | | |
|---|---|---|---|
| common | pUC57-in-b | GGA TGT GCT GCA AGG CGA | 41 |
| | hCH2-in-b | CCA GGA GTT CAG GTG CTG | 42 |

Primer for amplification of CDR and adjacent framework

| | | | |
|---|---|---|---|
| VH3-1 | pC3x-f | GCA CGA CAG GTT TCC CGA C | 43 |
| | VH3-1-b | TTACCTGGTGCCTGTCTG | 44 |
| VH3-2 | VH3-1-b-rc | CAGACAGGCACCAGGTAA | 45 |
| | VH3-2-b | GCGTGAGATGGTGAAGCG | 46 |
| VH3-H3 | VH3-2-b-rc | CGCTTCACCATCTCACGC | 47 |
| | VH-3-b | CAGAGTACCTTGTCCCCA | 48 |
| VL-1 | VH-3-rc-B | TGGGGACAAGGTACTCTG | 49 |
| | VL-2-f-rc | GTAAATCAGGAGCTTAGGAG | 50 |
| VL-2 | VL-2-f | CTCCTAAGCTCCTGATTTAC | 51 |
| | VL-3-f-rc | GCAGTAATAGTCAGCCTCG | 52 |
| VL-3 | VL-3-f | CGAGGCTGACTATTACTGC | 53 |
| | pC3x-b | AAC CAT CGA TAG CAG CAC CG | 54 |
| VK-1 | VH-3-rc-B | TGGGGACAAGGTACTCTG | 55 |
| | VK-2-f-rc | ATAGATGAGCAGGCGTGG | 56 |
| VK-2 | VK-2-f | CCACGCCTGCTCATCTAT | 57 |
| | VK-3-f-rc | ACAATAGTAAACTGCGAAGTC | 58 |
| VK-3 | VK-3-f | GACTTCGCAGTTTACTATTGT | 59 |
| | pC3x-b | AAC CAT CGA TAG CAG CAC CG | 60 |

Primer for assembly of VH or VL/VK fragment

| | | | |
|---|---|---|---|
| VH3 | pC3x-f | GCA CGA CAG GTT TCC CGA C | 61 |
| | VH-3-b | CAGAGTACCTTGTCCCCA | 62 |
| VL1 | VH-3-rc-B | TGGGGACAAGGTACTCTG | 63 |
| | pC3x-b | AAC CAT CGA TAG CAG CAC CG | 64 |
| VK3 | VH-3-rc-B | TGGGGACAAGGTACTCTG | 65 |
| | pC3x-b | AAC CAT CGA TAG CAG CAC CG | 66 |

Primer for amplification of final scFv

| | | | |
|---|---|---|---|
| common | pC3x-f | GCA CGA CAG GTT TCC CGA C | 67 |
| | pC3x-b | AAC CAT CGA TAG CAG CAC CG | 68 |

Sequencing Primer

| | | | |
|---|---|---|---|
| common | omp seq | AAGACAGCTATCGCGATTGCAG | 69 |

### Example 8: Library Structure and Phage Display Selection

Library rescue and panning were performed according to previously described methods [PLoS One. 2015;10: 1-18, Korean Patent Laid-open Publication No. 2016-0087766]. Specifically, 1 mL of sub-library E. coli stock was incubated in 400 mL of SB medium containing ampicillin and 2% filter-sterilized glucose. When the cell density at 600 nm reached 0.7 (OD600=0.7), cells were centrifuged, resuspended in 400 mL of SB medium containing 100 μg/mL ampicillin and infected with a helper phage VSCM13 (10¹² pfu) for 1 hour with gentle shaking(80 rpm) at 37° C. Then, kanamycin (70 μg/mL) was added and the bacteria were incubated overnight at 30° C. On the next day, the culture incubated overnight was centrifuged and phages were precipitated from the supernatant by dissolving 4% (w/v) PEG 8000 (Sigma Aldrich) and 3% (w/v) NaCl (Duchefa Biochemie). After incubation on ice for at least 30 minutes, the precipitated phages were collected by centrifugation, resuspended in 10 mL of PBS and centrifuged again to remove bacterial debris. Phages were re-precipitated from the supernatant as described above and resuspended in 2 mL of PBS, and glycerol was added to a final concentration of 15% and mixed thoroughly. The finally obtained phage library was frozen in aliquots of 10¹³ cfu with liquid nitrogen and stored at −80° C.

For selection of phages for a target antigen, the immunotube was coated with the target antigen contained at a concentration of 1 μg/mL to 10 μg/mL in PBS and blocked with PBST (mPBST) containing 3% nonfat dry milk for 1 hour. Then, mPBST containing 1013 cfu of a phage display library was added to the antigen-coated immunotube. After incubation at 37° C. for 1 to 2 hours, the tube was washed 2 to 5 times for one round and 5 to 10 times for the next round with PBST to remove unbound phages. The bound phages were eluted with 1 mL of 100 mM triethylamine for 5 min, transferred to a new 50 mL tube and neutralized with 0.5 mL of 1 M Tris (pH 7.4). TG1 E. coli cells (OD600=0.7, 8.5 mL) were infected with the neutralized phages, and 1 μL of the infected bacterial culture was diluted in 1 mL of SB medium. For output titering, 10 μL of the diluted culture and 100 μL of the diluted culture were plated on 100 mm LB-ampicillin agar plates. The remaining culture was grown overnight on 150 mm LB-amp plates(LB-amp plate) containing 100 ug/mL ampicillin and 2% glucose. On the next day, the culture was harvested and grown by adding cells (>10⁹) to 20 mL of SB medium containing ampicillin until OD600 reached 0.7, and a helper phage VCSM13 (10¹¹ pfu) was added thereto. Cells were infected at 37° C. for 1 hour with gentle stirring (120 rpm) and incubated overnight at 30° C. with stirring at 200 rpm in a medium containing 70 μg/mL kanamycin. On the next day, the culture was centrifuged, and 5×PEG precipitation buffer (20% [w/v] PEG-8000 and 15% [w/v] NaCl) was added to the phage-containing supernatant for precipitation, followed by incubation on ice for 30 minutes. The precipitated phages were harvested by centrifugation, and the phage pellets was resuspended in 300 μL of 1× PBS and used in subsequent rounds of panning. For input titering, 1μL of 10⁻⁷ diluted phage was added to 100 μL of mid-log phase E. coli cells and incubated at room temperature for 1 hour. The infected bacteria were plated on LB-ampicillin agar plates and incubated overnight at 37° C.

### Example 9: ELISA and Dot Blot Assay

Individual colonies selected from the panning output were incubated in an SB medium containing ampicillin in 96-well microtiter plates with stirring until cloudy. Then, IPTG[isopropyl β-D-1-thiogalactopyranoside (Duchefa Biochemie)] was added to each well at 1 mM, and the plates were incubated at 30° C. with stirring overnight. On the next day, the plates were centrifuged, the supernatant was discarded, and the cell pellets were resuspended in 40 μL of cold 1× TES buffer [50 mM Tris (Generay Biotech), 1 mM EDTA (Ameresco), 20% sucrose (Sigma Aldrich), pH 8.0)]. Subsequently, 60 μL of cold 0.2× TES buffer was added thereto. After standing on ice for 30 minutes, the plates were centrifuged and the supernatant containing periplasmic extract (PPE) was obtained. The supernatant was screened for antigen-binding scFv by ELISA. Then, 96-well ELISA plates (Costar 3690, Corning) were coated with PBS containing antigen at 1 to 10 μg/mL and blocked with mPBST at room temperature for 1 hour, and 25 μL of PPE was added to the wells of the blocked ELISA plates. After standing at room temperature for 1 hour, the ELISA plates were washed 3 times with 1× PBST, and 25 μL of HRP-conjugated anti-HA antibody (Santa Cruz Biotechnology) diluted 1:3,000 in mPBST was added to each well. After standing at room temperature for 1 hour, the plates were washed 5 times with 1× PBST, and 25 μL of TMB (tetramethylbenzidine) was added to each well for detection. The reaction was stopped by 25 μL of 1N H2504, and the absorbance at 450 nm was measured using a microtiter plate reader.

For dot blot assay, random E. coli clones were selected from the library and incubated in 96-well plates, and periplasmic extracts (PPE) were obtained by the method described above. Then, 1 μL of PPE was applied onto a nitrocellulose membrane (Whatman # 10401196) and dried. The membrane was blocked with mPBST for 1 hour and allowed to bind to HRP-conjugated secondary anti-HA antibody diluted 1:3,000 in mPBST through reaction for 1 hour. The membrane was rinsed 3 times with PBST and detected using an ECL solution (Abfrontier; LF-QC0101) and an X-ray film.

### Example 10: Surface Plasmon Resonance (SPR) Analysis

SPR analysis was performed using a Biacore 3000 system (Biacore, GE Healthcare). An antigen in sodium acetate(pH 4.0^{˜}5.5) was immobilized on a CM5 chip at a flow rate of 5 μL/min (target RU: 800 to 1200) according to the standard amine coupling protocol provided by the manufacturer and infected with purified scFv antibodies at six different concentrations. To obtain kinetic parameters and dissociation constants, data were fitted to a Langmuir 1:1 binding model or a 1:1 drifting baseline model by using BiaEvaluation software.

### Test Example 1: Previous Antibody Library Construction and Sequence Analysis

1-1: Construction and Analysis of Antibody Library (OPALS)

In a previous study, a synthetic human antibody library (hereinafter, referred to as “OPALS”), which has excellent functional diversity and is composed of antibodies with similar or superior properties to those of natural human antibodies, was constructed (PLoS One. 2015;10: 1-18, Korean Patent Laid-open Publication No. 2016-0087766). A process of constructing the conventional antibody library will be described briefly. In the scFv library, germline variable genes IGHV3-23 and IGKV3-20, or IGHV3-23 and IGLV1-47 are linked by a (Gly-Gly-Gly-Gly-Ser)3 and used as a framework. The CDRs are designed with non-combinatorial diversity to have sequence diversity similar to human antibody CDRs by characteristic analysis and simulation of the CDR sequences of known human antibodies.

Specifically, the framework sequences used herein are shown in Table 8 below. In the amino acid sequences of Table 8 below, X means any amino acid, a CDR is underlined, and a linker is bolded. Meanwhile, an improved antibody library, which will be described below, also used the same framework sequences at an amino acid level.

TABLE 8

| Framework | Sequence |
|---|---|
| (IGHV3- | N′- |
| 23)- | EVQLLESGGGLVQPGGSLRLSCAASGFTFSXXXXXWVRQ |
| linker- | APGKGLEWVXXXXXRFTISRDNSKNTLYLQMNSLRAEDT |
| (IGKV3- | AVYYCAKXXXXXWGQGTLVTVSSGGGGSGGGGSGGGGSE |
| 20) | IV |
| | LTQSPGTLSLSPGERATLSCXXXXXWYQQKPGQAPRLLI |
| | YXXXXXGIPDRFSGSGSGTDFTLTISRLEPEDFAVYYCX |
| | XXXXFGQGTKVEIK-C′ (SEQ ID NO: 70) |
| (IGHV3- | N′- |
| 23)- | EVQLLESGGGLVQPGGSLRLSCAASGFTFSXXXXXWVRQ |
| linker- | APGKGLEWVXXXXXRFTISRDNSKNTLYLQMNSLRAEDT |
| (IGLV1- | AVYYCAKXXXXXWGQGTLVTVSSGGGGSGGGGSGGGGSQ |
| 47) | SV |
| | LTQPPSASGTPGQRVTISCXXXXXWYQQLPGTAPKLLIY |
| | XXXXXGVPDRFSGSKSGTSASLAISGLRSEDEADYYCXX |
| | XXXFGGGTKLTVL-C′ (SEQ ID NO: 71) |

Human immunoglobulin sequences were downloaded from IMGT database(http://imgt.org), and CDR sequences were extracted. Then, CDR sequences of human antibody germline immunoglobulin genes from V-base(http://www2.mrc-Imb.cam.ac.uk/vbase/alignments2.php) were compared and analyzed with mature CDR sequences extracted from the IMGT database. As a result, (i) the germline CDR sequence, which is the closest to each mature CDR sequence, was found, and the position, type and frequency of mutations occurring in each mature CDR sequence were investigated, and (ii) the frequency of each germline CDR used in the mature human antibody repertoires was investigated.

Meanwhile, as for CDR-H3 and CDR-L3, it is difficult to identify the germline sequences due to VDJ recombination and mechanisms such as junctional flexibility, P-addition, or N-addition. Thus, as for CDR-L3, 2 or 3 amino acids at the terminus of CDR-L3 were designed by simulating the sequence frequency at the corresponding positions in CDR-L3 of the mature human antibodies. For the design of CDR-H3, the sequences were simulated by first dividing the CDR-H3 sequences of the mature human antibody repertoire by length and analyzing the frequency of use of amino acids at each position for each length.

Then, after the CDR sequences were simulated based on the amino acid frequency, sequences with sites for N-glycosylation, aspartate isomerization, asparagine deamidation, non-enzymatic cleavage or oxidation were minimized or excluded.

Each of the CDRs designed by the method described above has low diversity (^{˜}10³ unique sequences), but when they are combined, an antibody library with excellent functionality can be constructed. The CDR repertoires constructed by the method described above were assembled into a final scFv library through a series of overlap-extension (OE) PCRs and transformed to E. coli to construct an antibody library having >10⁹ individual clones.

1-2: Sequence Analysis of Antibody Library (OPALS)

Next generation sequencing (NGS) of the library was performed to assess the accuracy with which the CDR design was reflected in the antibody library constructed in Test Example 1-1. Millions of CDR sequences were analyzed and compared with the designed sequences (Table 9).

TABLE 9
Next-generation sequencing of library CDRs

| CDR- | H1 | H2 | H3 | K1 | K2 | K3 | L1 | L2 | L3 |
|---|---|---|---|---|---|---|---|---|---|
| Analyzed sequence (×10⁶) | 4.75 | 4.70 | 4.53 | 2.58 | 2.31 | 2.52 | 2.14 | 2.12 | 2.09 |
| In frame % | 91.3 | 89.7 | 90.3 | 92.2 | 91.6 | 93.2 | 91.5 | 78.7 | 90.7 |
| Accordance design length % | 89.4 | 88.4 | 85.6 | 87.1 | 82.3 | 90.7 | 84.6 | 56.2 | 89.4 |
| Accordance sequence length % | 80.9 | 52.0 | 51.8 | 62.4 | 70.3 | 74.3 | 56.2 | 47.6 | 67.2 |
| In frame before proofreading^{a} % | 85.7 | 50.0 | 67.9 | 53.3 | 44.4 | 66.7 | 38.5 | 31.3 | 66.7 |
| Design coverage %^{b} | 100 | 100 | 97.3 | 99.8 | 100 | 99.9 | 100 | 100 | 100 |

^{a}Percentage of in-frame CDR sequences before the proofreading panning was estimated by Sanger sequencing of 12-28 sequences per CDR.
^{b}Percentage of the designed CDR sequences was established by NGS.

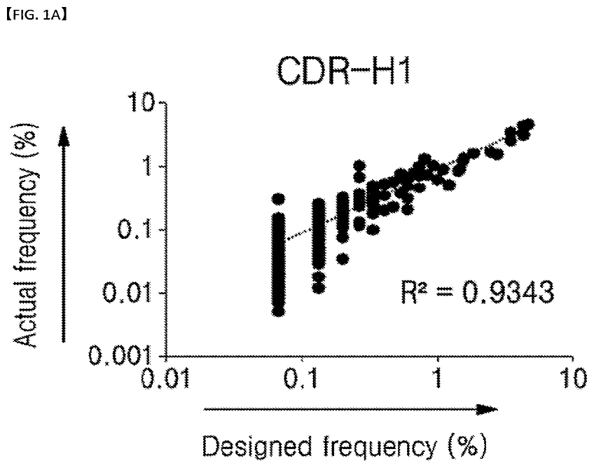

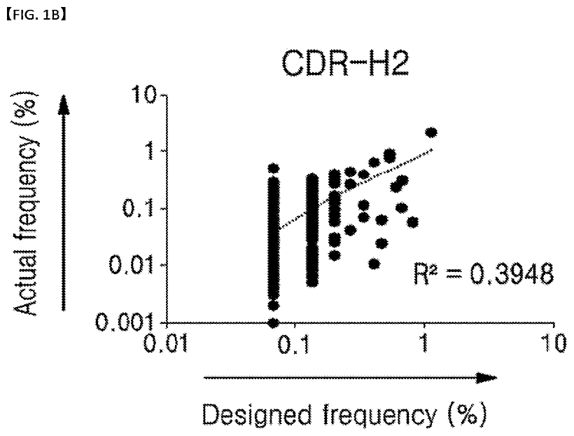

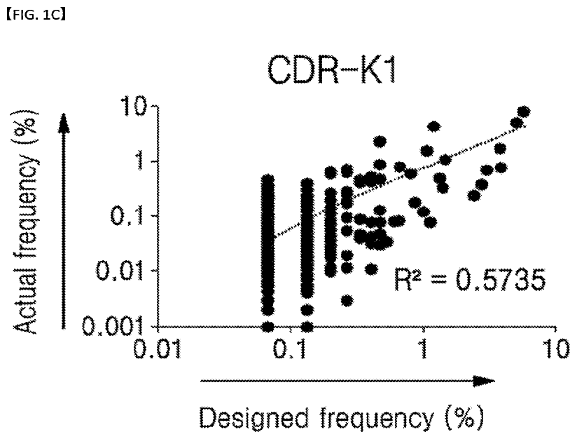

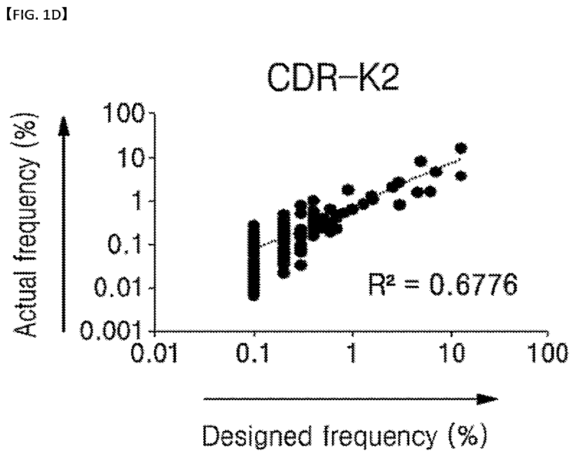

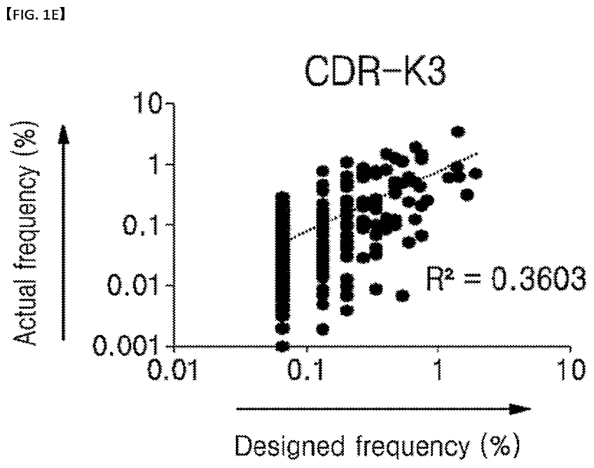

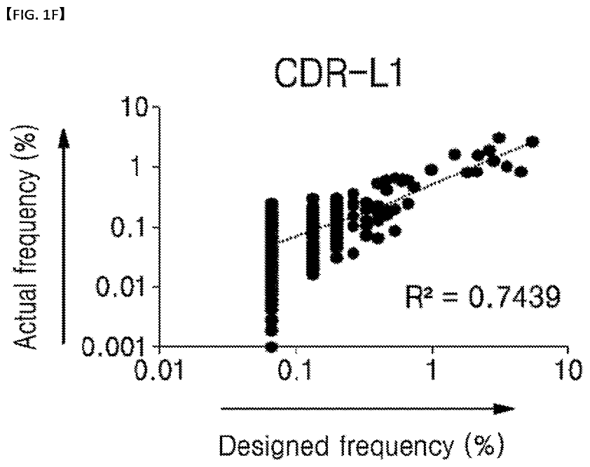

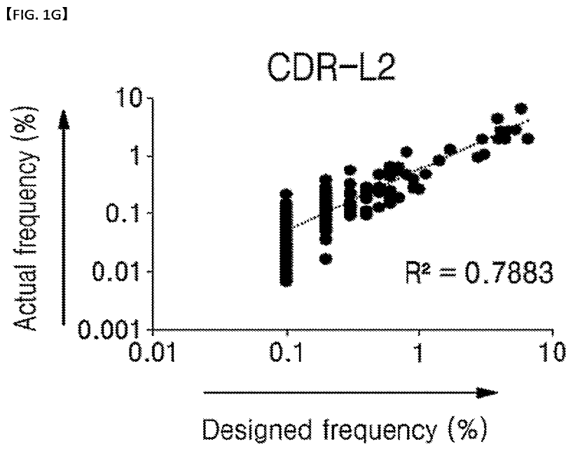

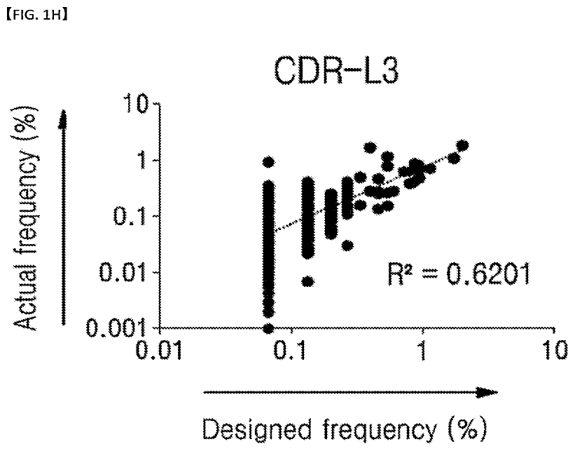

The designed sequences were nearly completely covered in the constructed library and the frequency of each designed CDR sequence was also represented in the actual library although, for long CDRs, a majority of the unique sequences occur only once or twice in all of the designed sequences and the coefficients of determination r² are relatively low (FIGS. 1A to 1H). Further, the corresponding library was found to contain many low-frequency CDR sequences not matching any of the designed sequences due to synthesis errors, including non-functional sequences with nucleotide insertions or deletions that cause frameshifts. The proofreading panning of the single-CDR libraries using anti-HA antibody removed the non-functional CDR sequences, and the percentage of functional in-frame CDR sequences was about 90% to 93% after the proofreading panning compared with 31% to 86% before the proofreading panning. The percentage of CDR-L2 was only 79%, which is likely due to inaccurate annealing during the overlap extension PCR. The percentage of CDR sequences with the designed lengths was about 82% to 91% (about 56% for CDR-L2). Overall, about 75% of V_{H}, V_{K} and V_{λ} sequences were functional domains without stop codons, and the percentage of functional scFv clones in the library was estimated to be approximately 55%.

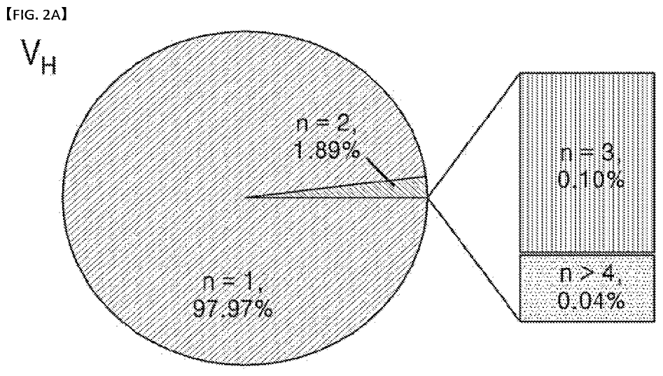

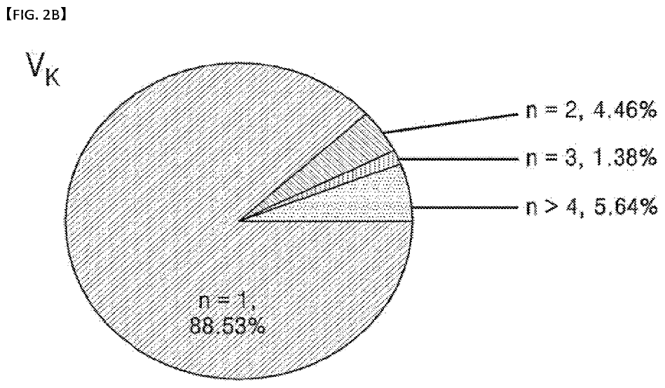

Then, unique domains out of various domains were analyzed from NGS data. Approximately 1.3×10⁶ domain sequences of each of the heavy chain, kappa light chain and lambda light chain without stop codons were analyzed, and 98% of VH, 89% of V_{K} and 98% of Vλ sequences were non-redundant (FIGS. 2A to 2C). The percentages of the numbers of different domain sequences among all the sequences were 99%, 92% and 99% for VH, V_{K}, and Vλ, respectively. Meanwhile, these values were comparable with the previously reported CDR-H3 sequence uniqueness of 97% to 98% for other antibody libraries, and suggest that the redundancy among scFv clones in the unselected library is not significant.

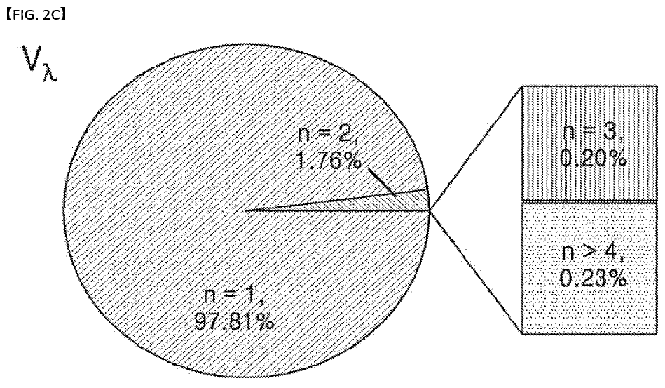

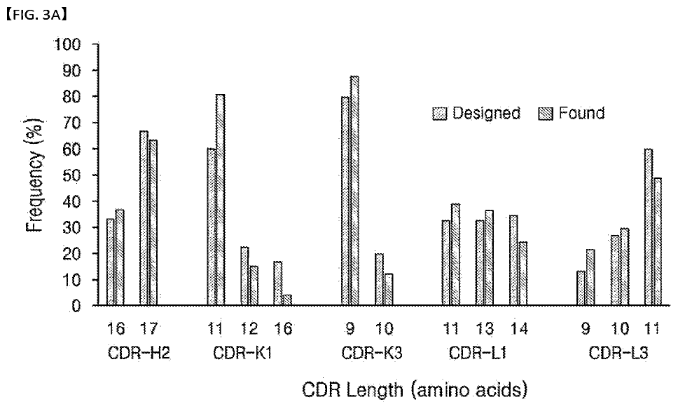

The distribution of CDR length, particularly of CDR-H3 length, in the constructed library was different from the design, with shorter length CDRs conspicuously overrepresented when compared with longer CDRs (FIGS. 3A and 3B). This is probably in part because of the inaccuracy during the oligonucleotide array synthesis that introduced frameshift and premature stop codons. These errors are more likely to occur during the synthesis of longer CDRs and most of them are removed during the proofreading panning of the single-CDR libraries using anti-HA-tag antibody. Therefore, it is considered that more of the longer CDRs were removed from the library. Also, it is possible that scFvs with shorter CDRs were preferentially selected and amplified by the panning against anti-HA-tag antibody.

Then, the similarity of the library CDR sequences to the natural CDR sequences was assessed by analyzing the number of amino acid differences in each CDR sequence from the closest germline CDR sequences. Because the CDRs were designed to simulate the natural somatic hypermutation (SHM) patterns, it was assumed that the designed sequences are highly nature-like. Therefore, the average numbers of mutations per CDR sequence were compared between the designed and the natural CDR sequences (Table 10).

TABLE 10
Average numbers of amino acid differences in CDR sequences
from closest human germline CDR sequences

| CDR^{a} | H1 | H2 | | K1 | | | K2 | K3 | |
|---|---|---|---|---|---|---|---|---|---|
| Length | 5 | 16 | 17 | 11 | 12 | 16 | 7 | 9 | 10 |
| Design^{b} | 0.75 | 2.14 | 2.55 | 1 | 1.18 | 1.85 | 0.47 | 0.73 | 0.65 |
| Non-design^{c} | 1.84 | 4.3 | 3.67 | 2.77 | 3.44 | 3.82 | 2.01 | 1.55 | 1.45 |
| Natural^{d} | 0.82 | 2.09 | 2.37 | 1.08 | 1.29 | 0.93 | 0.49 | 0.72 | 0.54 |

| CDR^{a} | L1 | | | L2 | L3 | | |
|---|---|---|---|---|---|---|---|
| Length | 11 | 13 | 14 | 7 | 9 | 10 | 11 |
| Design^{b} | 1.03 | 1.1 | 0.98 | 0.61 | 0.83 | 1.18 | 0.7 |
| Non-design^{c} | 2.82 | 3.22 | 2.75 | 1.85 | 1.68 | 2.11 | 1.45 |
| Natural^{d} | 0.95 | 1.1 | 1 | 0.66 | 0.88 | 1.14 | 0.75 |

^{a}CDR-H3 was not analyzed because there were no germline sequences to compare with
^{b}Designed CDR sequences as described in Examples
^{c}The library CDR sequences that did not match any designed sequence but had the correct length.
^{d}Natural human CDR sequences with corresponding length retrieved from the IMGT database.

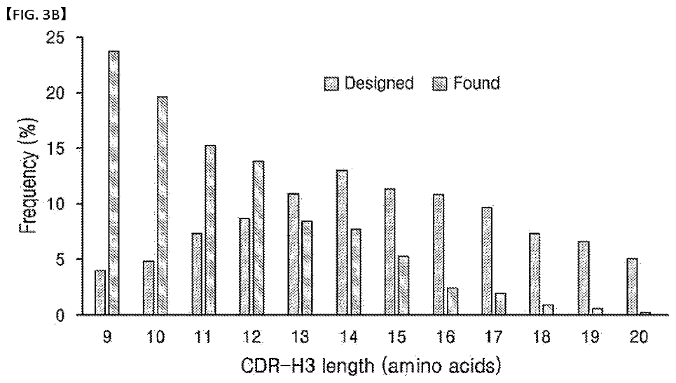

When the library CDR sequences (non-designed CDR sequences) that did not match any of the designed sequences due to synthesis errors were analyzed, the average number of amino acid differences from the closest germline CDR sequence was different by only 1-2 amino acids on average from that of the designed CDR sequences. These results suggest that the CDR sequences of the library contain only small numbers of mutations from the human germline CDR sequences, and are highly similar to the CDR sequences of natural human antibodies. As for CDR-H3, the amino acid distribution at each position was analyzed (FIG. 4). Similar amino acid distribution patterns were found among the CDR-H3s of natural human antibodies, the simulated repertoire and the constructed library, demonstrating high nature-likeness of the library CDRs.

The post-translational modifications of proteins may affect the functions and physicochemical properties of the proteins. These include N-glycosylation, aspartate isomerization, asparagine deamidation, peptide bond cleavage, and amino acid side chain oxidation. Aspartate (Asp) isomerization frequently occurs particularly in an Asp-Gly sequence, asparagine (Asn) deamidation frequently occurs particularly in an Asn-Gly sequence, peptide bond cleavage frequently occurs particularly in an Asp-Pro sequence, and amino acid side chain oxidation frequently occurs particularly in cysteine and methionine. Thus, CDR sequences containing these motifs were excluded from the design. As expected, the frequency of appearance of undesirable post-translational modification motifs in the CDRs was significantly lower than that in native human antibody CDRs, with the exceptions of CDR-H1 and CDR-L2, which are short with 5 and 7 amino acids, respectively, and have fewer PTM motifs in natural human antibodies (Table 11). Assuming that the PTM motifs occur independently in different CDRs, the probability of at least one PTM motif(s) occurring in scFv sequences in the library is estimated to be approximately 20% to 30%. Also, 70% to 80% of the clones are assumed not to have PTM motifs, whereas only 24% to 27% of scFvs from natural sources do not have PTM motifs.

TABLE 11
Percentages of post-translational modification motifs
in CDRs of library and natural human antibodies

| Motif | CDR- | H1 | H2 | H3 | K1 | K2 | K3 | L1 | L2 | L3 |
|---|---|---|---|---|---|---|---|---|---|---|
| Asp-Gly | Library | 0.05 | 0.31 | 0.66 | 0.03 | 0.34 | 0.10 | 0.32 | 3.41 | 0.34 |
| | Natural | 0.08 | 10.79 | 6.45 | 6.03 | 0.32 | 0.09 | 0.28 | 0.30 | 1.98 |
| Asn-Gly | Library | 0.04 | 0.62 | 0.32 | 0.05 | 0.63 | 0.12 | 0.60 | 4.51 | 0.32 |
| | Natural | 0.02 | 5.96 | 3.03 | 4.54 | 0.53 | 0.50 | 0.06 | 0.12 | 7.61 |
| Asp-Pro | Library | 0.01 | 0.16 | 0.25 | 0.04 | 0.08 | 0.10 | 0.04 | 0.20 | 0.05 |
| | Natural | 0.00 | 2.40 | 10.13 | 0.00 | 0.40 | 0.14 | 0.11 | 0.00 | 0.66 |
| N-glyc. | Library | 0.66 | 1.91 | 2.77 | 1.35 | 1.46 | 1.00 | 1.97 | 0.94 | 1.30 |
| | Natural | 0.59 | 0.78 | 7.73 | 0.61 | 0.07 | 1.69 | 6.74 | 0.30 | 8.20 |
| Met | Library | 0.35 | 1.03 | 1.80 | 0.90 | 0.55 | 0.29 | 1.78 | 0.70 | 1.70 |
| | Natural | 0.06 | 2.72 | 9.04 | 0.88 | 0.11 | 12.36 | 0.39 | 0.18 | 0.86 |
| Cys | Library | 0.30 | 1.96 | 0.49 | 1.30 | 0.64 | 0.77 | 2.10 | 0.87 | 1.29 |
| | Natural | 0.35 | 17.50 | 1.46 | 0.92 | 0.18 | 1.19 | 0.61 | 0.24 | 1.98 |
| Total | Library | 1.36 | 5.44 | 5.87 | 3.60 | 3.58 | 2.32 | 6.16 | 10.48 | 4.81 |
| PTM | Natural | 1.09 | 36.70 | 32.92 | 12.55 | 1.23 | 15.56 | 8.08 | 1.13 | 20.70 |

### Test Example 2: Construction of Improved Novel Antibody Library (OPALT)

In order to construct a novel antibody library improved from the antibody library (OPALS) synthesized in Test Example 1, the following test was performed.

2-1: Confirmation of Correlation Between Germline CDR and Panning Efficiency

IGHV3-23 germline (VH3-23, DP47) used as heavy chain framework in a conventional antibody library (OPALS) is well known to be able to bind to protein A. Thus, panning of OPALS against protein A is expected to enrich the number of rapidly growing or highly displayed clones irrespective of antigen specificity or CDR sequences. Therefore, in order to check whether the CDR sequences affect the panning results, the conventional antibody library (OPALS) was panned 3 rounds against protein A, and the sequences of the panning output was analyzed using an Illumina Miseq platform. Millions of variable region sequences were decoded by paired-end sequencing and translated into amino acid sequences, and CDR sequences were extracted using a self-developed Python program.

Based on the above process, it was analyzed how the percentages of five CDRs except for CDR-H3 in the library derived from the germline CDRs, which is the basis for the CDR design, changes before and after panning against protein A. As a result of analysis, it was confirmed that there was no significant change in the relative percentage of the germline origin of the CDR sequences except for CDR-H2 before and after panning against protein A. This is considered to mean that the CDR sequences based on the various germline CDR sequences are well accepted with respect to the IGHV3-23, IGKV3-20 and IGLV1-47 frameworks, and are normally expressed and displayed.

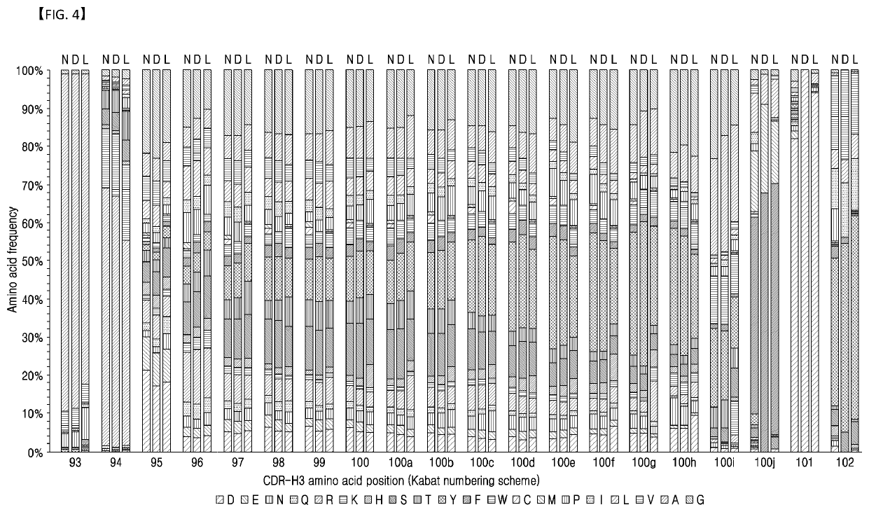

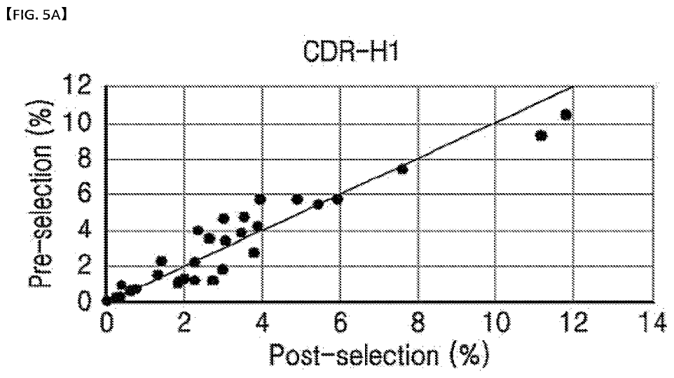

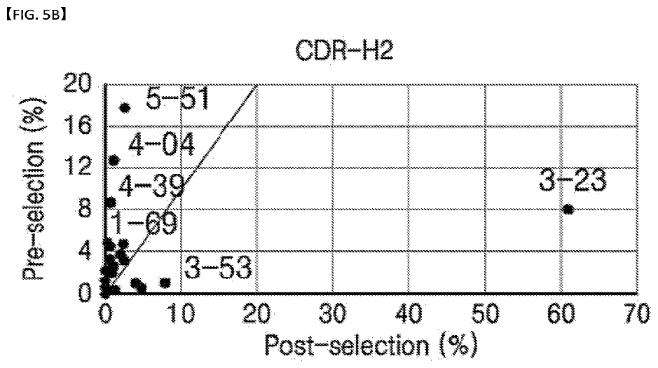

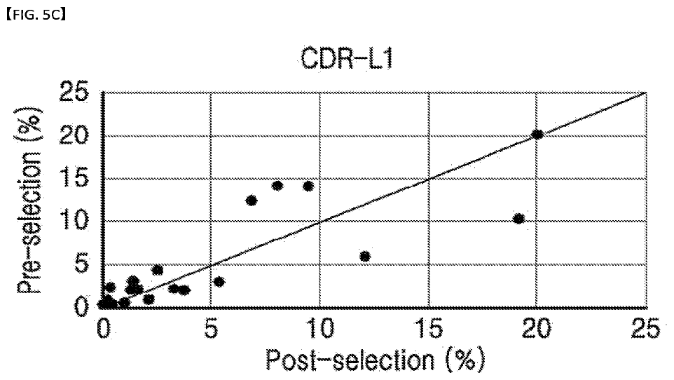

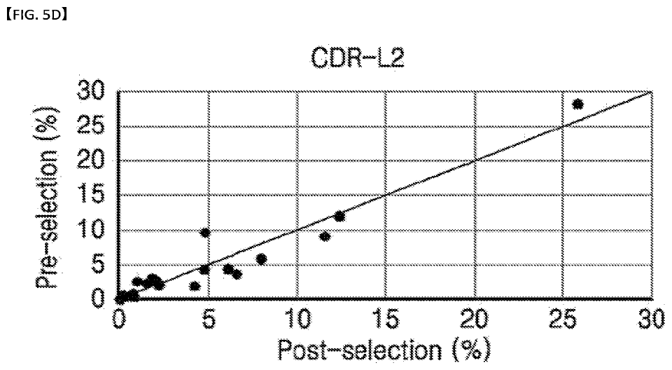

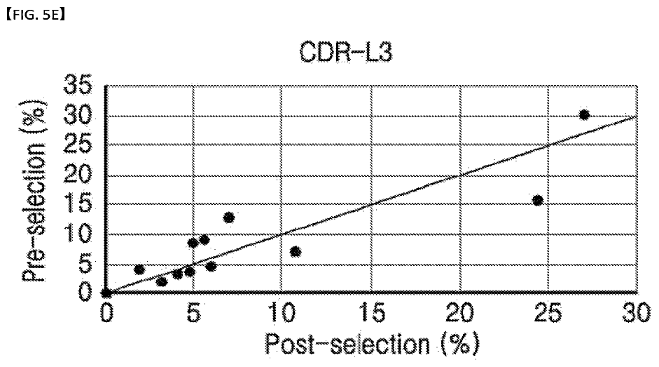

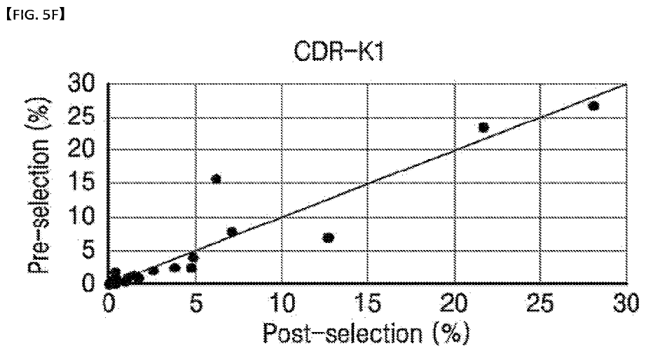

Meanwhile, as for CDR-H2, it was confirmed that the CDR sequences derived from the germline sequences belonging to VH3 were highly enriched after panning, whereas the CDR sequences derived from the germline CDR sequences belonging to VH1, VH4 or VH5 were significantly reduced after panning. was confirmed (FIGS. 5A to 5H).

Based on the above result, it can be seen that the germline of the CDR sequences needs to be considered in order to improve the panning efficiency when an antibody library is designed, and the improved antibody library (OPALT) was designed generally by using CDRs of all variable gene families, and CDR-H2 was designed avoiding the sequences derived from the VH1, VH4 or VH5 family.

2-2: Design Method of CDR-H3 Sequences Based on NGS Analysis Result and Enrichment Scores of CDR-H3 Sequences

Based on the result of Test Example 2-1, when a novel antibody library (OPALT) was designed, a strategy was established that constructed a library by grafting the VH3-derived sequences of CDR-H2 onto the IGHV3-23 framework while using the IGHV3-23, IGKV3-20 and IGLV1-47 frameworks. According to the above strategy, it is expected that a library with an enhanced display level of antibody fragments will be constructed.

Then, as for the design of the CDR-H3 sequences, the CDR-H3 sequences of the conventional antibody library was not designed based on the germline sequences and thus cannot be analyzed in the same way as in other CDRs. Instead, the relative frequencies of individual CDR-H3 sequences were measured before and after panning against protein A, and an enrichment score for each sequence was calculated therefrom. There were large differences between the frequencies of individual sequences in the library, which was considered to be related to the suitability of the sequences. In other words, among the sequences designed to have the same frequency, the higher frequency of some clones in the library prior to panning may be due in part to their high suitability in the E. coli host. Therefore, the frequency was considered when the enrichment frequency was calculated.

Therefore, for the design of CDR-H3, the NGS data and enrichment scores for CDR-H3 were organized for each length (from 9 to 20 amino acids), and then the CDR-H3 sequences and enrichment scores were tabulated in a spreadsheet file and analyzed. An enrichment score ESᵢ for any specific CDR-H3 sequence i in the antibody library is calculated using the following formula.

$$Enrichment score ( ES ) = {log}_{2} \frac{{n}_{i - post} / {N}_{post}}{{n}_{i - pre} / {N}_{pre}} \times \sqrt{\frac{{n}_{i - post} + {n}_{i - pre}}{median ( {n}_{post} ) + median ( {n}_{pre} )}} \qquad [ Equation 1 ]$$

Herein, Nₚᵣₑ and Nₚₒₛₜ are the total numbers of CDR-H3 reads through NGS analysis of the antibody library before and after panning, respectively, nᵢ₋ₚᵣₑ and median(nₚₒₛₜ) are medians of read numbers of the specific CDR-H3 sequence before and after panning, respectively.

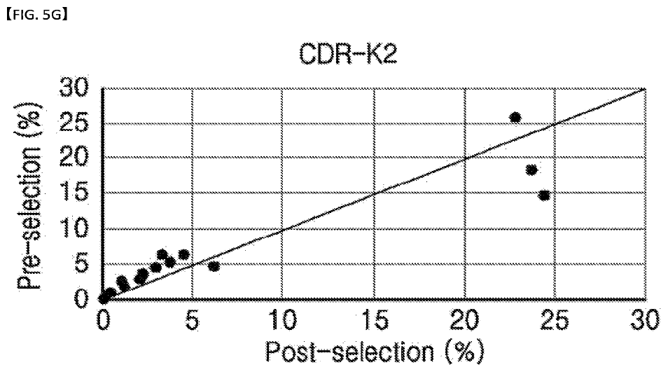

As a result of sequences analysis before and after panning of CDR-H3, it was confirmed that there was a significant change in the amino acid frequency at each position of CDR-H3 after panning (FIG. 6). Specifically, it was confirmed that Val, Trp and Tyr have significant disadvantages at positions H96/H97, H95/H96/H97 and H98, respectively, whereas Asp, Gly and Pro are significantly preferred at positions H98, H95 and H98, respectively. Based on the above result, it can be seen that the amino acid sequences of CDR-H3 are an important determinant of the panning efficiency of phage antibody clones, and it is possible to design the CDR sequences, particularly CDR-H3 sequences, in order to construct a synthetic antibody library with more efficiently enriched clones.

Based on the above result, the enrichment data were used to train a machine learning model by using the enrichment scores for individual sequences and amino acid residues at respective positions as input values. To minimize potential overestimation of enrichment scores for clones with low read counts, only sequences with nᵢ₋ₚᵣₑ or nᵢ₋ₚₒₛₜ greater than 10 for each length were taken into account. Herein, randomly selected 70% of a dataset was used as a training set and the remaining 30% was used as a validation set.

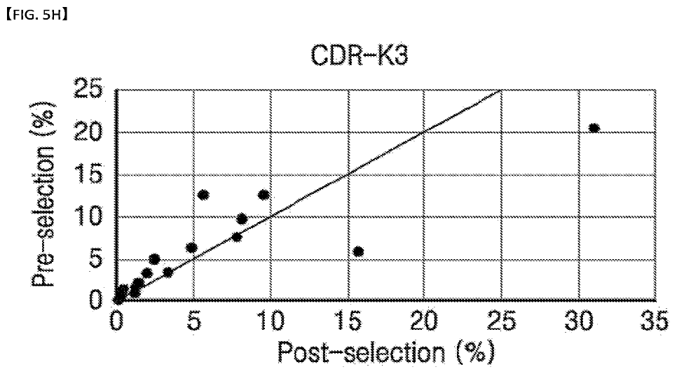

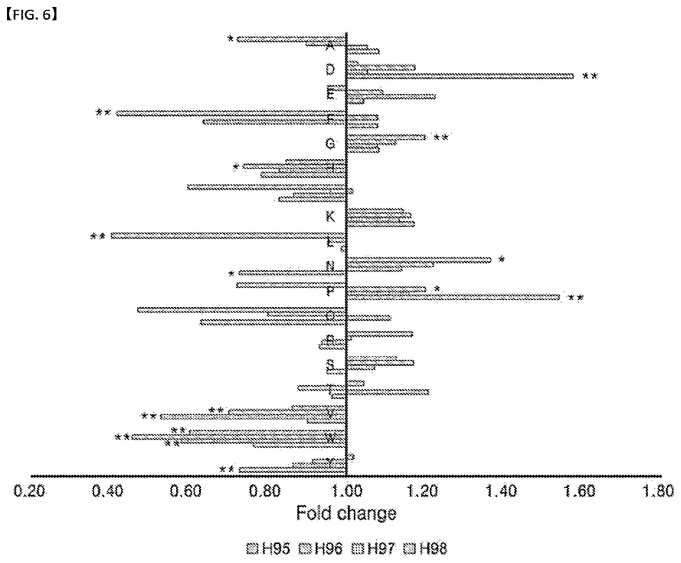

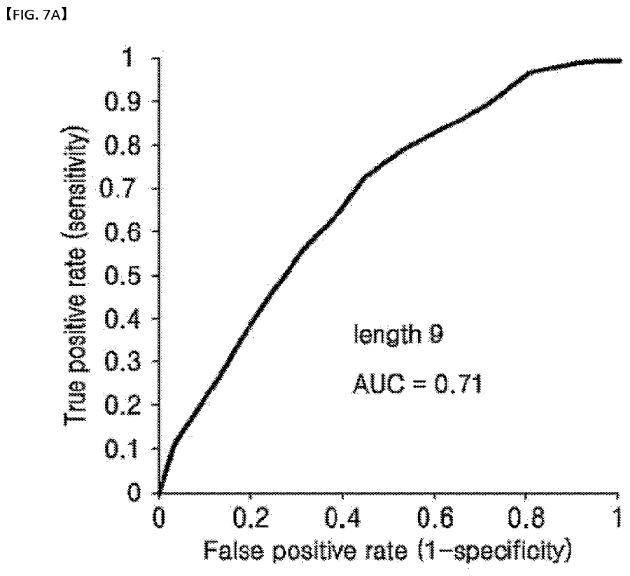

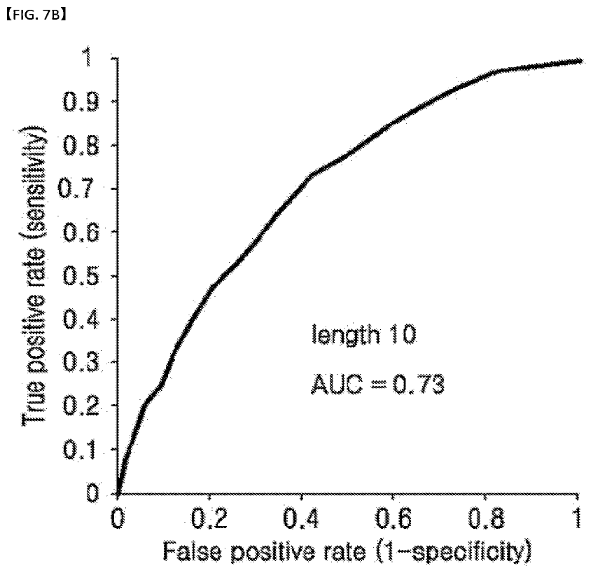

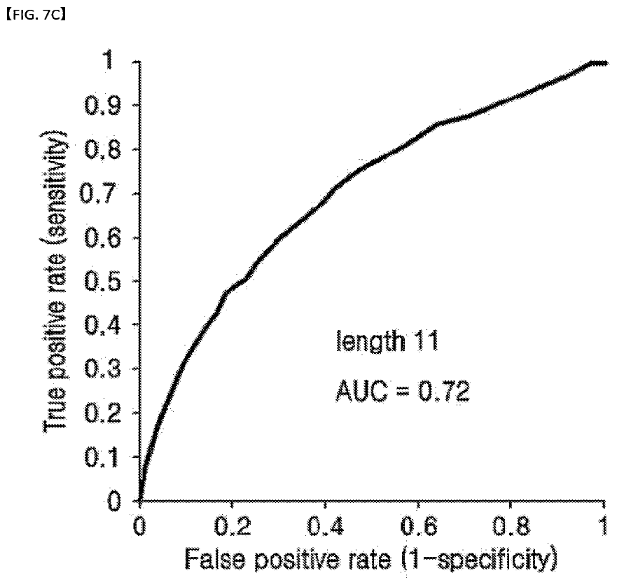

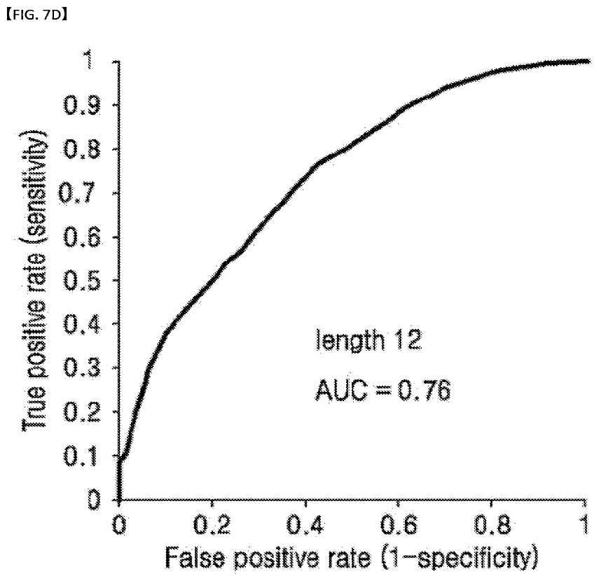

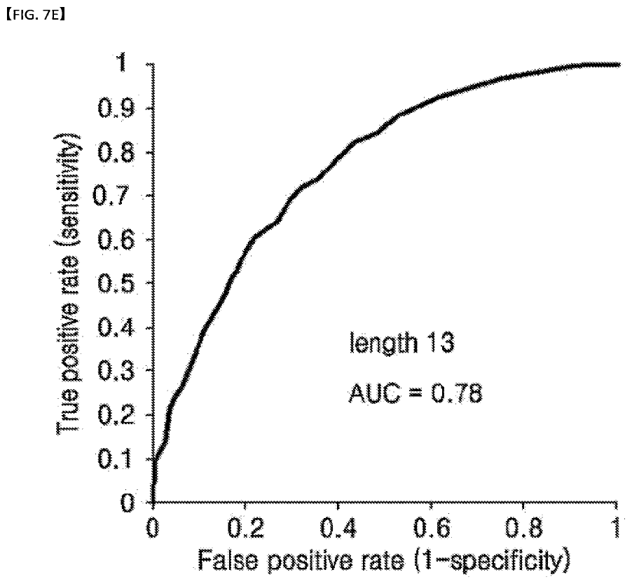

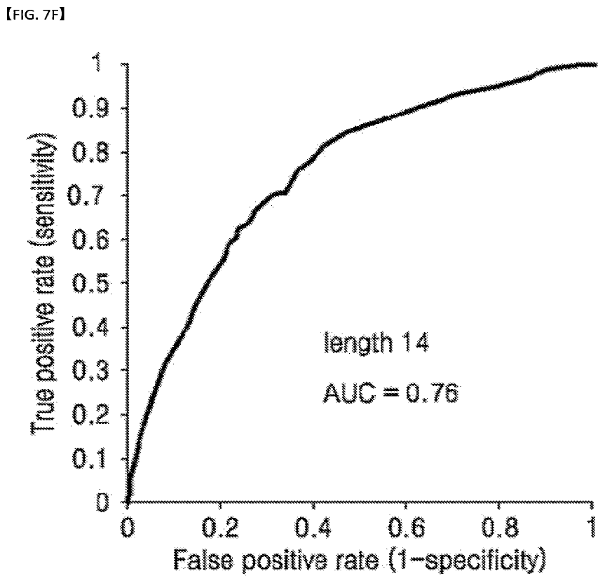

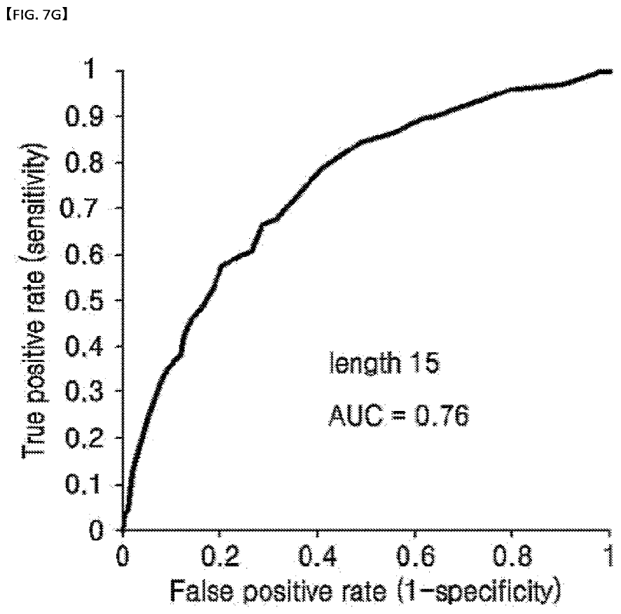

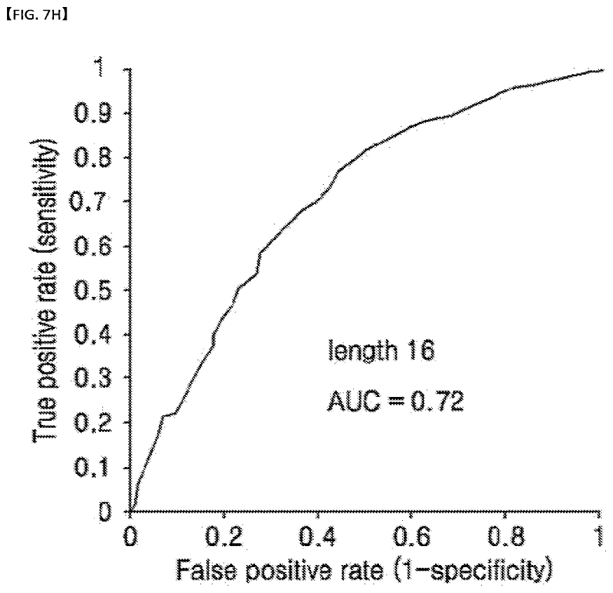

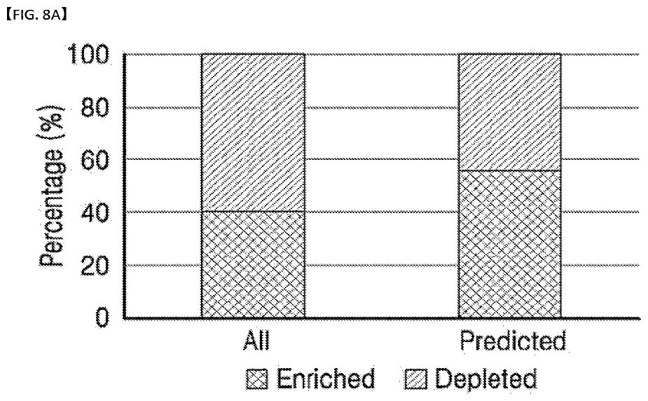

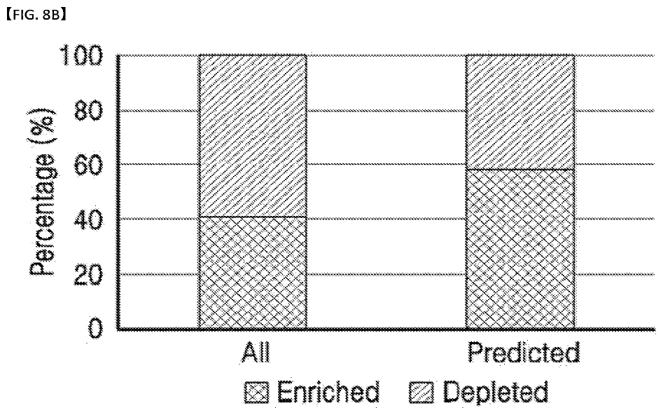

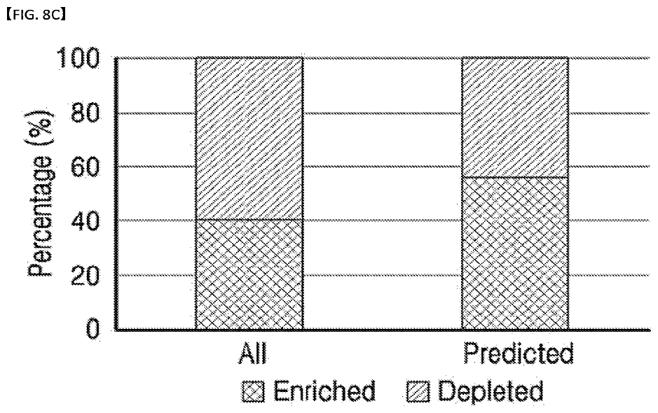

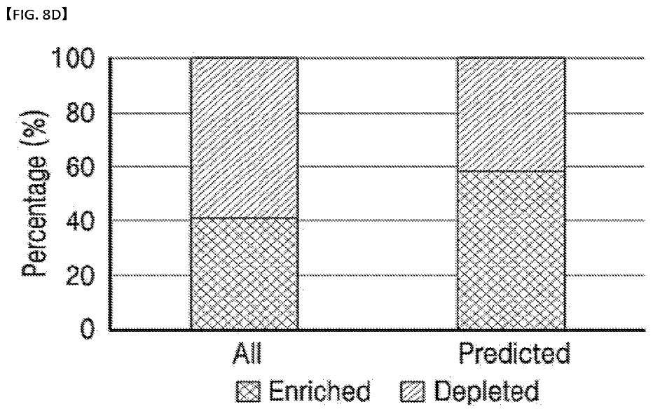

The enrichment scores of sequences predicted by the machine learning model in the validation set had an intermediate correlation with the enrichment scores calculated from the actual NGS results (FIGS. 7A to 7H). One of the reasons for the intermediate correlation may be that the display and production level of an antibody cannot be determined only by CDR-H3 sequences. However, after panning, ^{˜}40% of the CDR-H3 sequences were enriched (ES>0), but when only clones with predicted ES>0 were taken into account, the percentage increased to ^{˜}60% (FIGS. 8A to 8H). Therefore, it can be seen that machine learning performed as described above can be a useful tool for predicting the suitability of the CDR-H3 sequences.

The CDR sequences for the novel antibody library were designed as described above, i.e., by simulating the frequencies of amino acids of natural human antibodies for CDR-H3 and simulating the use of germline genes and somatic hypermutation of natural human antibodies for the other CDRs. Sequences with PTM motifs were excluded after simulation. To select sequences expected to be enriched by panning, the machine learning model was applied to the CDR-H3 sequences designed as described above. Among the CDR-H3 sequences selected by machine learning, excessively or repetitively appearing single amino acids were excluded.

Then, the designed CDR sequences were de-immunized in silico using the netMHCIIpan-3.1 program. Specifically, 8 amino acids from adjacent framework regions were added to both sides of the CDR amino acid sequences. The binding strength of the 9-aa peptide fragments overlapping each other in the constructed fragment sequences to MHC class II was evaluated using the netMHCIIpan-3.1 program. For example, in the case of CDR-H1 consisting of 5 amino acids, evaluation was made on the binding strength of 13 peptide fragments from the 9-aa peptide fragment consisting of 1^{st} to 9^{th} amino acids to the 9-aa peptide fragment consisting of 13^{th} to 21^{st} amino acids to the 21-aa fragment sequence in which 8 amino acids are added to each of both framework regions. Sequences predicted to have strong binding to one or more of 20 common MHC class II alleles were excluded from the design.

Nucleic acid sequences of gene frameworks IGHV3-23, IGKV3-20 and IGLV1-47 were newly designed by introducing synonymous mutation to remove codons rarely used in humans and E. coli, maintain GC content between 50% and 70% within a 30-bp sliding window, and minimize cross-priming between similar sequences among framework sequences. A total of 27,426 oligonucleotide sequences of 100-mer length were designed by reverse-translating the designed CDR amino acid sequences and adding the framework sequences to both sides.

2-3: Isolation of CDR-H3 by Length by PAGE

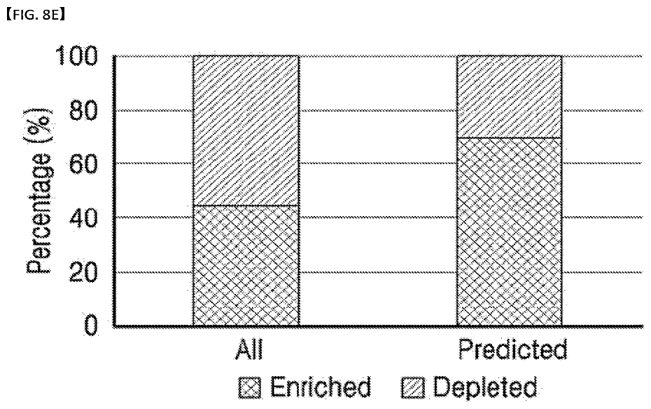

According to the analysis result of the conventional antibody library (OPALS) of Test Example 1-2, it has been mentioned that the short CDR-H3 appears excessively in the library (FIG. 3B), and, thus, a significant bias occurs in the CDR length distribution, which may cause degradation in the performance of the library. Accordingly, as for an improved novel antibody library (OPALT), it was determined to construct a plurality of sub-libraries each having a specific CDR-H3 length to minimize bias. Therefore, since the oligonucleotides encoding the designed CDR sequences were synthesized in parallel as a pool, it is necessary to isolate the CDR-H3-encoding oligonucleotides by their own length. To this end, the synthetic oligo pool and DNA amplified by PCR using a pair of CDR-H3-specific primers were purified by agarose gel electrophoresis. Then, the PCR-amplified CDR-H3 oligonucleotides were isolated by their own length on a 10% denaturing polyacrylamide gel. The denaturing conditions (8 M elements in the gel) were used to more clearly isolate DNA bands having different lengths by only 3 bases (FIG. 9). The DNA bands were visualized using SYBR Gold, which provides strong staining for single-stranded DNA than conventional ethidium bromide, and cleaved by length to obtain DNA by the method described above in Examples. The PAGE-purified CDR-H3 oligonucleotides were used as templates for PCR to amplify CDR-H3 having different amino acid lengths.

2-4: Construction of Novel Antibody Library (OPALT)

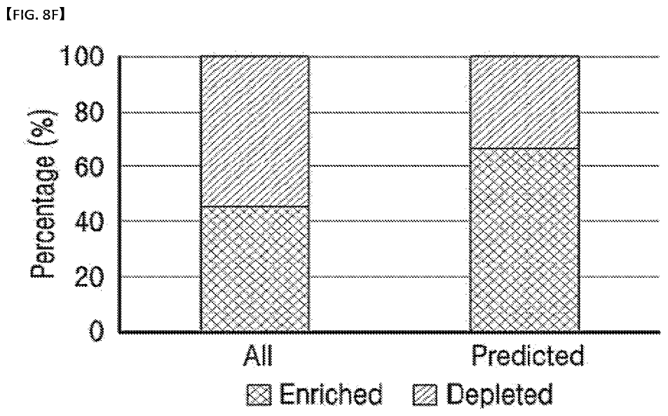

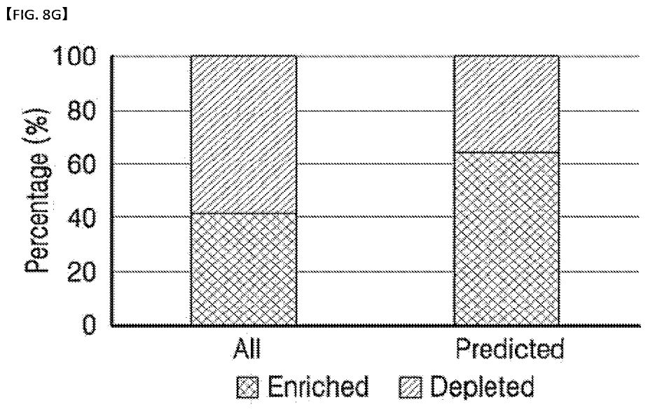

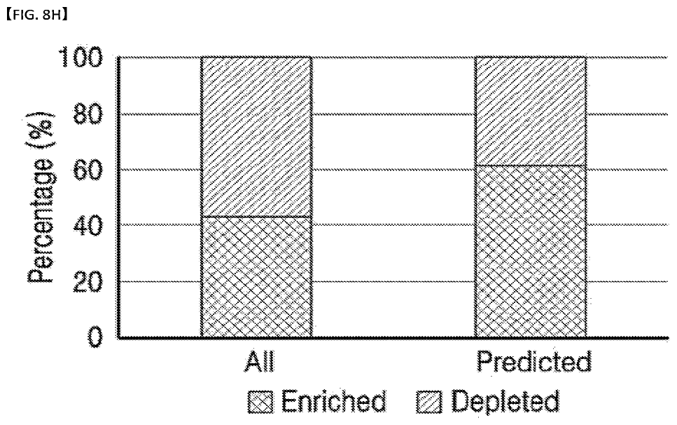

The designed CDR-encoding oligonucleotides were synthesized as described above and amplified by PCR (Table 1). Human germline immunoglobulin variable segments DP47(IGHV3-23), DPL3(IGLV1-47) and DPK22(IGKV3-20) were synthesized as frameworks of scFv libraries, cloned to pUC57 and pFcF vectors, and amplified by PCR as shown in Table 2. To suppress amplification of original template sequences in OE-PCR to be performed later, the framework sequences on the 5′-side of the CDR was obtained from the pUC57 construct and the 3′-side framework was obtained from the pFcF construct. The amplified CDR was grafted into the template scFv sequences amplified by overlap extension PCR (Table 3), and the product was ligated into the pComb3× phagemid vector. It was transformed into TG1 E. coli to construct a single CDR library (H1, H2, L1, L2, L3, K1, K2, K3) (FIG. 10B). As for CDR-H3, single-CDR libraries were constructed for each length (9 to 16 amino acids). The synthesized CDR-H3 oligonucleotide was amplified by PCR, and oligonucleotides having different lengths were isolated using a polyacrylamide gel. The isolated DNA was recovered from the polyacrylamide gel and combined with a template scFv framework to construct a single CDR-H3 library (VH3VL1/VH3VK3-H3 #1^{˜}#8) (FIG. 10A). The single library includes various heavy chain CDRs with ^{˜}10⁶ clones and various kappa/light chain CDRs with 10⁷ to 10⁸clones, and the CDR-H3 library is composed of 10⁸ clones. This single CDR library was subjected to one round of panning (except for H2 and L2 single CDR libraries that had undergone two rounds of panning) against protein A or protein L (Table 4). Protein A is well known to bind to an Fc region of heavy chain and to a variable domain of the human VH3 family. Unlike protein A, protein L binds to an antibody through light chain interaction and is limited to antibodies including a kappa light chain. Therefore, protein A and protein L were used to select productive scFv sequences with DP47-DPL3 (VH3VL1) and DP47-DPK22 (VH3VK3) frameworks, respectively.

Then, the proofread CDR sequences from the single-CDR library were used as templates for PCR amplification (Table 5). The amplified CDRs were assembled into V_{H} and V_{L}/V_{K} by OE-PCR (Table 6). The variable domains were further assembled into an scFv repertoire with six different CDRs by a series of OE-PCRs (Table 6), and the scFv DNA fragment cleaved with a restriction enzyme Sfil was ligated to the pCom3× phagemid vector cleaved with Sfil. (FIG. 10C). The ligated DNA was transformed to a TG1 E. coli strain by electroporation. Eight sub-libraries with different CDR-H3 lengths (9^{˜}16) were constructed for each of VH3VL1 and VH3VK3 scaffolds (VH3VL1 #1^{˜}#8 and VH3VK3 #1^{˜}#8, respectively). These sub-libraries were combined into two libraries (OPALT-λ with a DPL3 lambda light chain and OPALT-κ with a DPK22 kappa light chain) for phage rescue and panning experiments. Therefore, final scFv libraries with a total diversity of 1.1×10¹⁰ (OPALT-λ) and 3.4×10⁹ (OPALT-κ) individual clones, respectively, were obtained.

2-5: Confirmation of Percentage of In-frame CDR Sequences Increased After Proofreading Panning of Single CDR Library

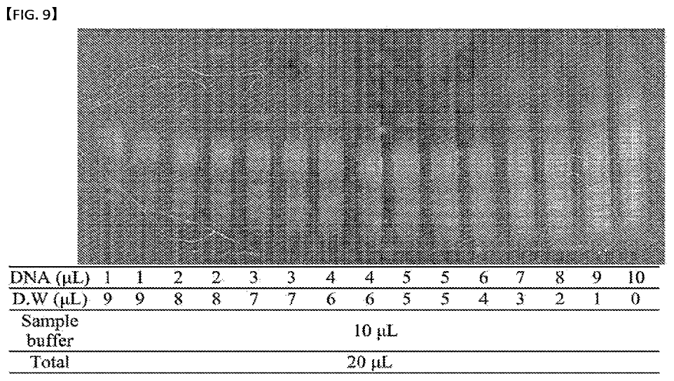

As described above, in order to remove the non-functional CDR sequences in the process of constructing the antibody library, one or two rounds of panning of the single-CDR library against protein A or protein L were performed. Then, dot blot assay was performed to check the result of panning. To this end, periplasm extracts of random scFv clones from the single-CDR library before and after panning selection were blotted onto a nitrocellulose membrane, and the presence of solubly expressed scFv in the extracts was detected by being bound to an anti-HA-HRP secondary antibody. As a result, the percentage of solubly expressed scFv clones was 23% to 86% before panning and increased to 82% to 100% after proofreading panning (FIG. 11).

Based on the above result, it can be seen that the array synthesis introduces a significant number of nucleotide insertions and deletions that cause frameshift and non-functional CDRs, and most of these sequences can be removed through proofreading panning.

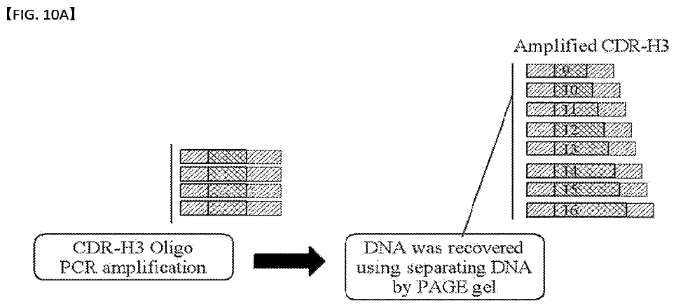

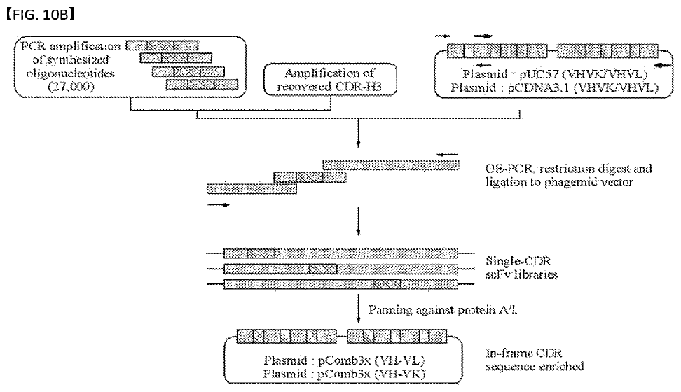

After the final scFv repertoire was assembled and transformed to E. coli, the sub-libraries constituting the final scFv libraries OPALT-λ and OPALT-κ represented about 50% and 40%, respectively, of the solubly expressed scFv clones (FIGS. 12A and 12B).

### Test Example 3: Confirmation of Functionality of Novel Antibody Library (OPALT)

3-1: Confirmation of Production of Antigen-specific Antibody

In order to verify the function of the improved novel antibody library (OPALT) constructed according to Test Example 2, OPALT, which is the library constructed according to the present disclosure, was compared with OPALS, which is one of the conventional libraries. Specifically, the libraries were panned using various antigens including various antigens frequently used as model antigens. The types of antigens used are as follows: Ag1, a non-disclosed antigen with a polyhistidine tag at the C terminus; CARS(Cysteinyl-tRNA synthetase); BCMA(B cell maturation agent) highly expressed on plasma cells; 140 kDa single-spanning membrane glycoprotein CD22 expressed on the surface of B cells; cell surface protein and adhesion molecule hNinj1(nerve injury-induced protein 1) involved in a neuroinflammatory response; AIMP1[ARS (aminoacyl-tRNA synthetase)-interacting multifunctional protein] that stimulates an inflammatory response after proteolytic cleavage in tumor cells; and SerRS(Seryl-tRNA synthetase).

Antigen-specific scFv clones were selected from the library after up to 4 rounds of panning. Colonies from the result of the 3rd or 4th round panning were screened by ELISA, and some colonies showing positive signals were sequenced (Table 11 and Table 12). As a result, in panning against Ag1, clones screened positive in OPALS-λ and OPALS-κ were 4% (4/94) and 2% (2/94), respectively, whereas OPALT-λ and OPALT-κ obtained 11% (10/94) and 26% (24/94) positive clones, respectively, and it was confirmed that a majority of the sequenced clones were unique. Further, as for BCMA, CD22 and hNinj1, fewer ELISA-positive clones were isolated in OPALS, but after panning with OPALT-λ, 90 (96%), 92 (98%) and 94 (100%) of positive clones were screened (Table 12).

TABLE 12
Result of 4th round panning and screening of
two types of libraries against each antigen

| | | | ELISA positive/ | Unique sequence/ |
|---|---|---|---|---|
| | Antigen | Library | Screening | Total sequence |
| 4^{th} round | Ag1 | OPALT-λ | 10/94 | 7/7 |
| | | OPALT-κ | 24/94 | 6/9 |
| | | OPALS-λ | 4/94 | 1/1 |
| | | OPALS-κ | 2/94 | 2/2 |
| | CARS | OPALT-λ | 92/94 | 8/8 |
| | | OPALT-κ | 91/94 | 2/10 |
| | | OPALS-λ | 94/94 | 3/8 |
| | | OPALS-κ | 89/94 | 5/9 |
| | hNinj1 | OPALT-λ | 94/94 | 2/6 |
| | | OPALT-κ | 93/94 | n.d |
| | | OPALS-λ | 8/94 | 2/3 |
| | | OPALS-κ | n.d | n.d |
| | BCMA | OPALT-λ | 90/94 | 5/11 |
| | | OPALT-κ | n.d | n.d |
| | | OPALS-λ | 1/47 | 1/1 |
| | | OPALS-κ | 2/47 | 2/2 |
| | CD22 | OPALT-λ | 92/94 | 3/8 |
| | | OPALT-κ | n.d | n.d |
| | | OPALS-λ | 27/94 | 2/8 |
| | | OPALS-κ | 12/94 | 2/6 |
| | SerRS | OPALT-λ | 80/94 | 4/5 |
| | | OPALS | 16/94 | 3/14 |
| | AIMP1 | OPALT-λ | 86/94 | 5/5 |
| | | OPALS | 66/94 | 5/8 |

Panning against CARS produced a high percentage of positive clones without a significant difference between the two libraries, and a high percentage of positive clones was also obtained from the third round panning. Also, all clones sequenced in the 4th round panning of OPALT-λ were unique (8/8), but the percentage of unique clones of the 4th round panning of the other library was slightly reduced. This result suggests that, due to the machine learning-based design of the CDR-H3 sequences expected to be efficiently enriched by phage display panning, scFv clones of OPALT can be enriched more uniformly during panning. In addition to CARS, the results of the 3rd round panning against Ag1 and hNinj1 were screened by ELISA, and unique sequences of a sufficient number of positive clones were obtained from the both libraries (Table 13).

TABLE 13
Result of 3rd round panning and screening of
two types of libraries against each antigen

| | | | ELISA positive/ | Unique sequence/ |
|---|---|---|---|---|
| | Antigen | Library | Screening | Total sequence |
| 3^{rd} round | Ag1 | OPALT-λ | 19/94 | 10/13 |
| | | OPALT-κ | 48/94 | 4/8 |
| | | OPALS-λ | 5/94 | 3/3 |
| | | OPALS-κ | 16/94 | 11/13 |
| | CARS | OPALT-λ | 91/93 | 7/8 |
| | | OPALT-κ | 91/94 | 3/8 |
| | | OPALS-λ | 91/94 | 5/10 |
| | | OPALS-κ | 46/94 | 10/12 |
| | hNinj1 | OPALT-λ | 52/94 | 4/6 |
| | | OPALT-κ | 91/94 | 2/5 |
| | | OPALS-λ | 4/94 | 1/4 |
| | | OPALS-κ | 66/94 | 3/7 |

OPALS was panned against SerRS and AIMP1 while a previous library was verified. Although there was no significant difference from the OPALS library in the number of unique sequences, OPALT was superior in the number of ELISA-positive clones.

3-2: Confirmation of Expression Frequency by Length of CDR-H3

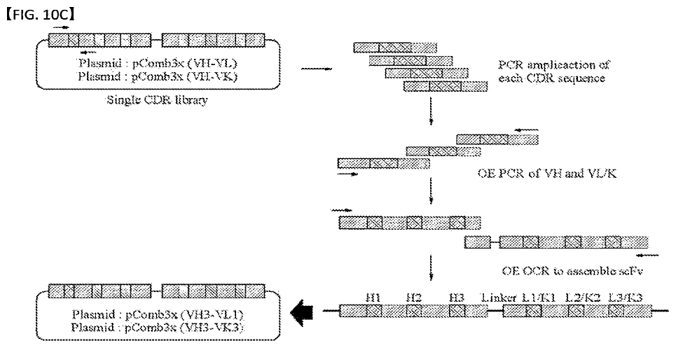

CDR-H3 of OPALT was designed to have a length of 9 to 16 amino acids, and the synthesized oligonucleotide pool for CDR-H3 of OPALT unlike OPALS was isolated by length before library construction. The frequency of appearance of CDR-H3s with different lengths in the sequences obtained by panning was analyzed (FIG. 13). As observed from the conventional antibody library (OPALS), scFvs with short CDR-H3s appeared more frequently in binding agents selected from OPALT, but long CDR-H3s with 14 and 15 amino acids were observed with a frequency of about 15%. The above result shows an improvement compared with OPALS in which all of CDR-H3s, which are not previously isolated by length and have different lengths, are combined, and errors during synthesis and more efficient enrichment of shorter CDR-H3 may be unfavorable for selection of long CDR-H3 sequences.

3-3: Confirmation of Affinity of OPALT-derived Clone

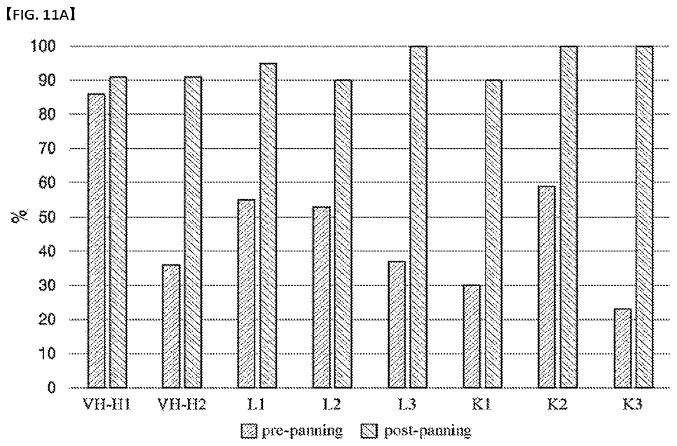

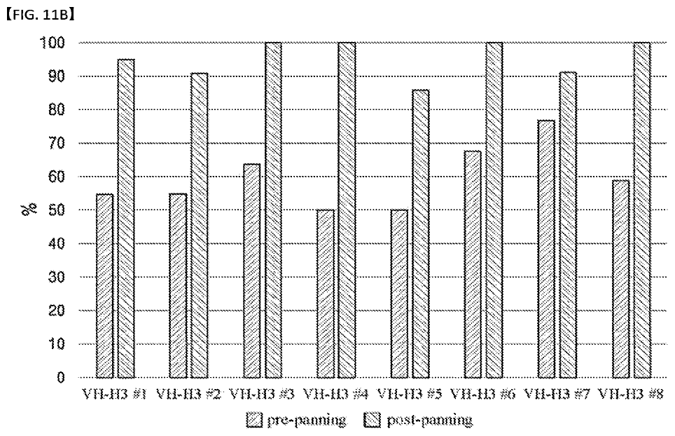

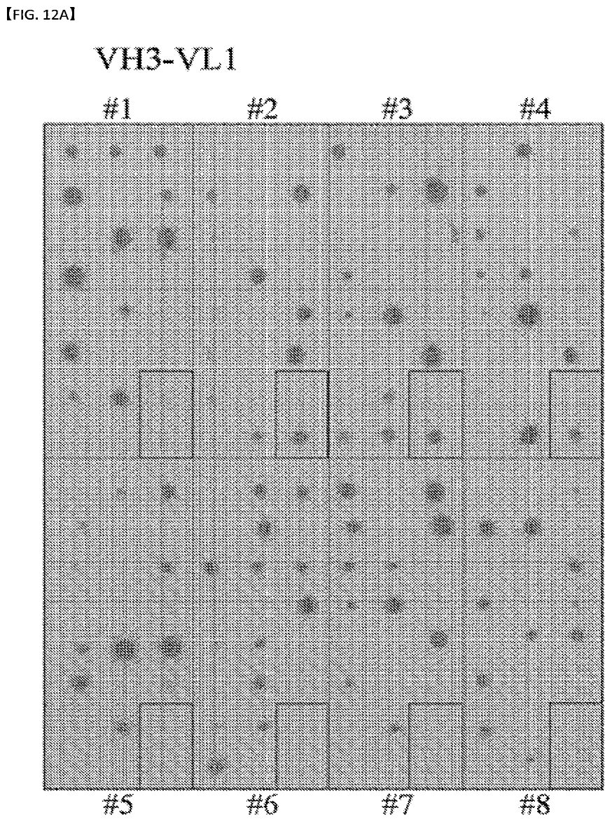

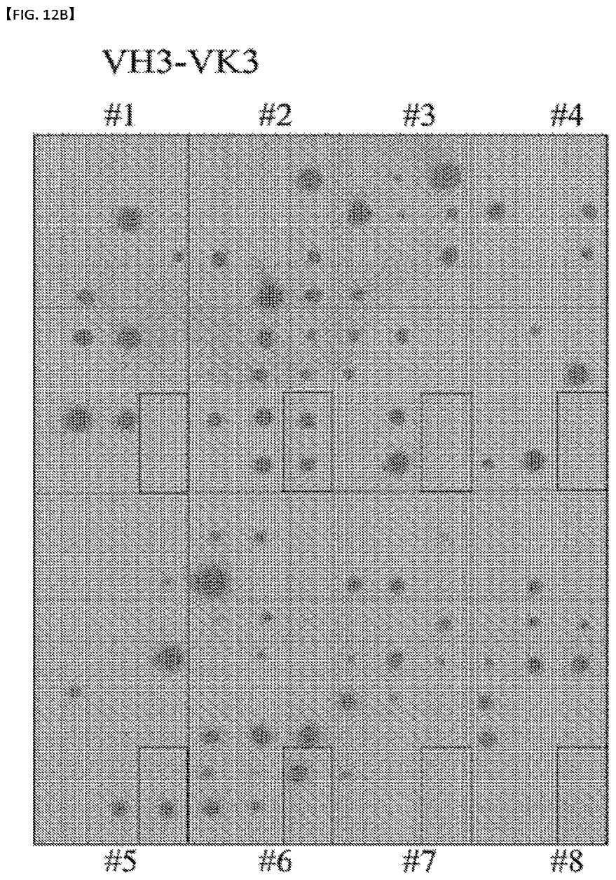

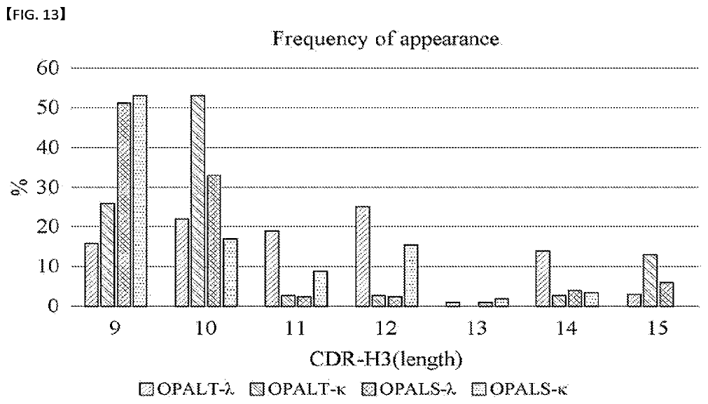

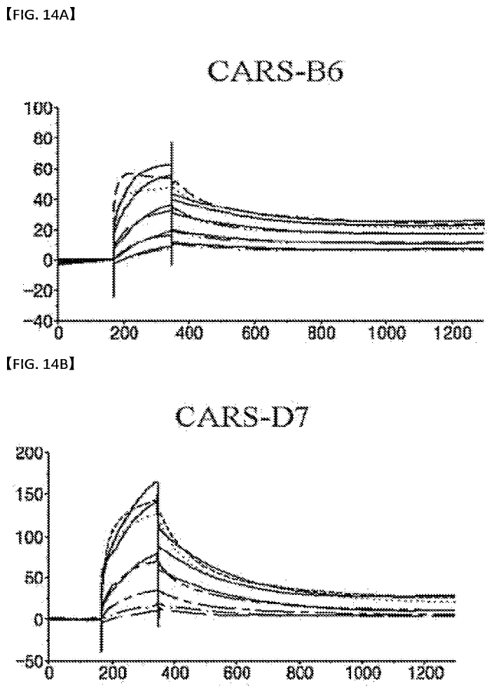

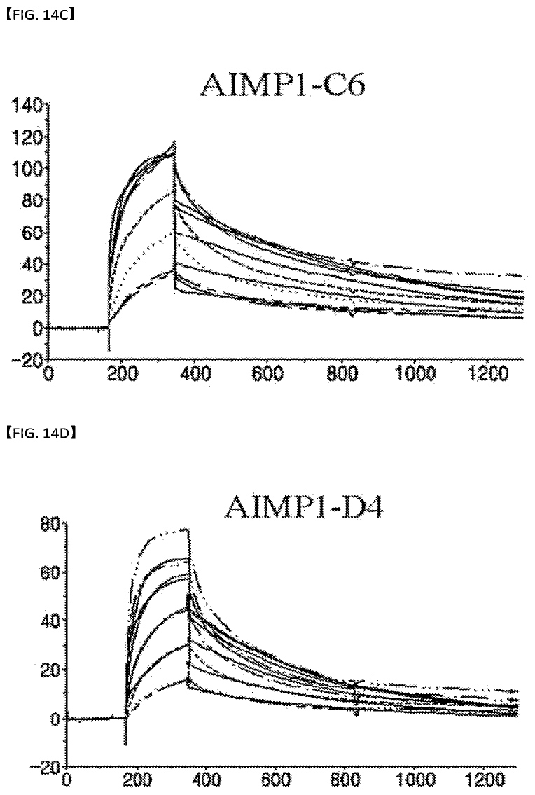

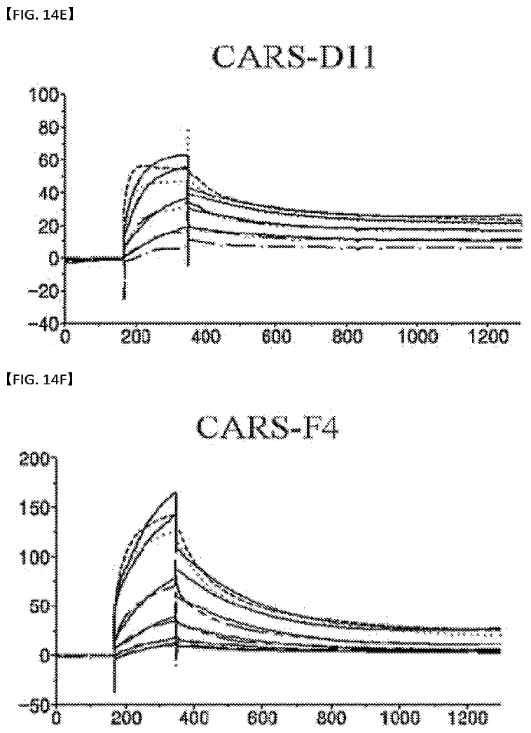

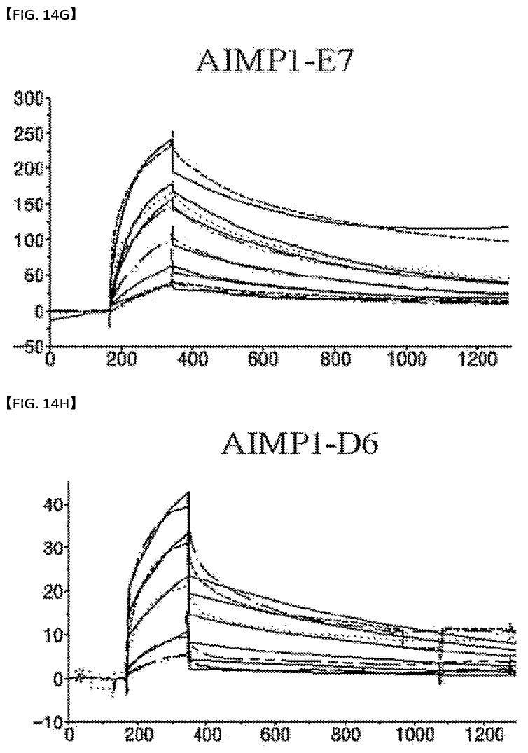

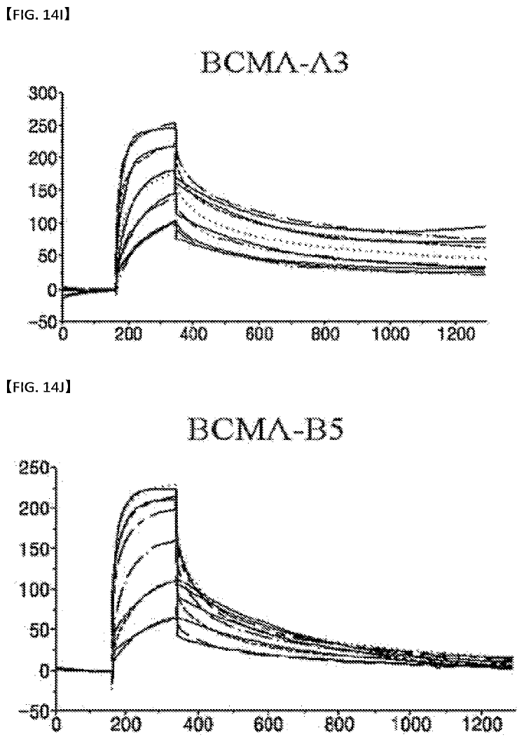

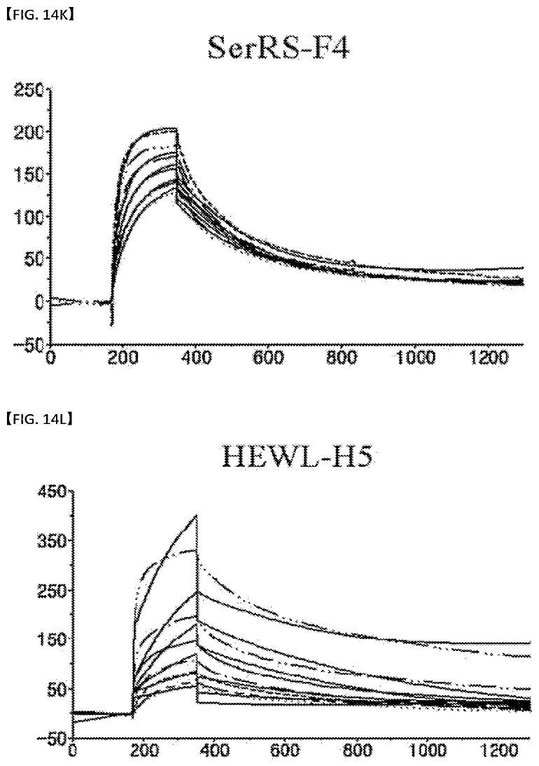

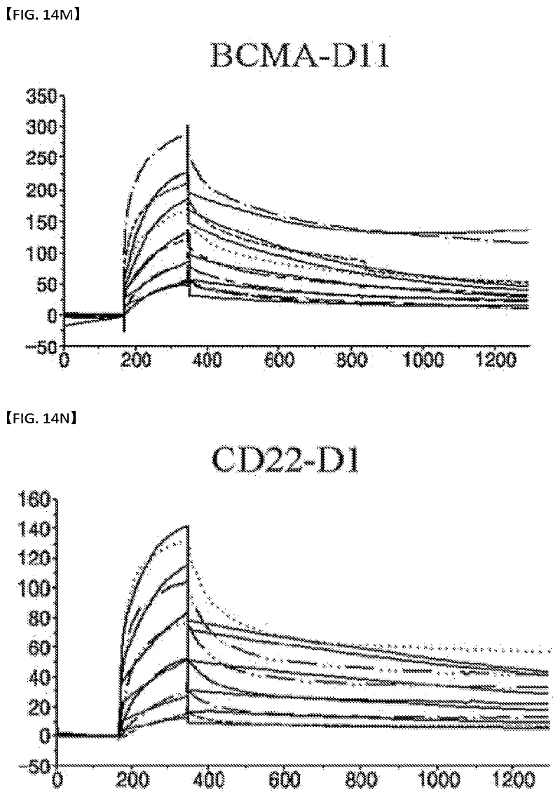

Some of the ELISA-positive scFv clones isolated from OPALT were analyzed by SPR to determine binding kinetics (FIGS. 14A to 14N, and Table 14). Specifically, scFv antibodies purified from a total of 13 E. coli host cells against five different antigens were analyzed using a Biacore 3000 device. The 13 scFv antibodies were found to have dissociation constants (K_{D}) ranging from 20 nM to 360 nM (mean K_{D}=84 nM).

TABLE 14
Binding kinetics of target-specific scFv
clones selected from OPALT (by SPR)

| Antigen-clone | kₒₙ (M⁻¹s⁻¹) | k_{off} (s⁻¹) | K_{D} (M) |
|---|---|---|---|
| CARS-B6 | 1.0 × 10⁵ | 4.2 × 10⁻³ | 4.1 × 10⁻⁸ |
| CARS-D7 | 1.6 × 10⁴ | 2.2 × 10⁻³ | 1.3 × 10⁻⁷ |
| CARS-D11 | 2.8 × 10⁴ | 2.3 × 10⁻³ | 8.2 × 10⁻⁸ |
| CARS-F4 | 3.6 × 10⁴ | 3.7 × 10⁻³ | 1.1 × 10⁻⁷ |
| BCMA-A3 | 8.7 × 10⁴ | 2.6 × 10⁻³ | 3.0 × 10⁻⁸ |
| BCMA-B5 | 7.5 × 10⁴ | 2.7 × 10⁻³ | 3.7 × 10⁻⁸ |
| BCMA-D11 | 2.4 × 10⁴ | 1.9 × 10⁻³ | 7.6 × 10⁻⁸ |
| AIMP1-C6 | 5.4 × 10⁴ | 1.5 × 10⁻³ | 2.8 × 10⁻⁸ |
| AIMP1-D4 | 3.2 × 10⁴ | 2.5 × 10⁻³ | 7.6 × 10⁻⁸ |
| AIMP1-E7 | 1.8 × 10⁴ | 2.0 × 10⁻³ | 1.1 × 10⁻⁷ |
| SerRS-D6 | 5.8 × 10⁴ | 1.1 × 10⁻³ | 2.0 × 10⁻⁸ |
| SerRS-F4 | 1.5 × 10⁵ | 4.0 × 10⁻³ | 2.7 × 10⁻⁸ |
| CD22-D1 | 1.4 × 10⁴ | 6.2 × 10⁻⁴ | 4.5 × 10⁻⁸ |

Also, most of the antibodies isolated from OPALT were found to have medium to high binding affinity, and two other ELISA-positive scFvs showed no binding activity in the SPR assay format. No antibody with a K_{D} value of 10⁻⁹ M or less among the analyzed clones was found, but these values once again supported the validity of the non-combinatorial CDR design approach. Most of association rates (kₒₙ) were about 10⁴ M⁻¹ s⁻¹, and dissociation rates (k_{off}) were about 10⁻³ s⁻¹, which seems to be in the range of expected values for typical monoclonal antibodies. Overall, it was confirmed that after panning against several antigens, OPALT can yield target-specific clones with K_{D} values in nanomolar range, and these are comparable to monoclonal antibodies obtained from other phage antibody libraries with similar or larger sizes or immunized animals.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A method of preparing an antibody library, comprising:
individually designing complementarity determining region (CDR) sequences of antibodies; and
synthesizing antibodies including the designed CDR sequences to prepare a library,
wherein, when designing the CDR sequences individually, heavy chain complementarity determining region 3 (CDR-H3) is optimized using enrichment scores of candidate CDR-H3 sequences.

2. The method of claim 1,
wherein the enrichment scores are predicted by a machine learning model, and
the machine learning model is trained by
a) setting at least one CDR-H3 sequence(s) as input values, and
b) setting, as result values, the enrichment scores calculated by measuring relative frequencies of the sequences before and after panning.

3. The method of claim 1,
wherein the enrichment scores are calculated using following Equation 1,
$$Enrichment score ( ES ) = {log}_{2} \frac{{n}_{i - post} / {N}_{post}}{{n}_{i - pre} / {N}_{pre}} \times \sqrt{\frac{{n}_{i - post} + {n}_{i - pre}}{median ( {n}_{post} ) + median ( {n}_{pre} )}} \qquad [ Equation 1 ]$$
[Nₚᵣₑ: Total number of NGS reads of a library including at least one candidate CDR-H3 sequence(s) before panning, Nₚₒₛₜ: Total number of NGS reads of the library after panning, nᵢ₋ₚᵣₑ: Number of reads of a specific CDR-H3 sequence i in the library before panning, nᵢ₋ₚₒₛₜ: Number of reads of the specific CDR-H3 sequence i in the library after panning, nₚᵣₑ: Set of read numbers of individual CDR-H3 sequences in the library before panning, nₚₒₛₜ: Set of read numbers of individual CDR-H3 sequences in the library after panning, median(nₚᵣₑ): Median of the nₚᵣₑ, and median(nₚₒₛₜ): Median of the nₚₒₛₜ].

4. The method of claim 1,
wherein, designing optimized sequences using the enrichment scores is calculating or predicting the enrichment scores of candidate CDR-H3 sequences to select candidate CDR-H3 sequences with the enrichment scores calculated or predicted to be above 0.

5. The method of claim 1,
wherein, when designing the CDR sequences individually, in the case of CDR-H2, sequences derived from the VH1, VH4 or VH5 family are excluded.

6. The method of claim 1,
wherein heavy chain complementarity determining region 1 (CDR-H1), heavy chain complementarity determining region 2 (CDR-H2), heavy chain complementarity determining region 3 (CDR-H3), light chain complementarity determining region 1 (CDR-L1), light chain complementarity determining region 2 (CDR-L2) and light chain complementarity determining region 3 (CDR-L3), which constitute the complementarity determining regions of the antibodies included in the antibody library, have polymorphism.

7. The method of claim 1,
wherein, when designing the CDR sequences individually, for CDR-H1, CDR-H2, CDR-L1 or CDR-L2,
the sequences therefor are designed by simulating i) an utilization frequency of each germline immunoglobulin gene, ii) a frequency of mutation into each of 20 amino acids by somatic hypermutations at each amino acid position, iii) a frequency of each CDR sequence length or iv) a frequency of each amino acid at each position calculated by analyzing a combination thereof, of CDRs of actual human-derived mature antibodies.

8. The method of claim 1,
wherein, when designing the CDR sequences individually, for CDR-L3,
a) 7 to 8 amino acid sequences from an N-terminus of the CDR are designed by simulating i) an utilization frequency of each germline immunoglobulin gene, ii) a frequency of mutation into each of 20 amino acids by somatic hypermutations at each amino acid position, iii) a frequency of each CDR sequence length or iv) a frequency of each amino acid at each position calculated by analyzing a combination thereof, of CDRs of actual human-derived mature antibodies, and
b) 2 to 3 amino acid sequences from a C-terminus of the CDR are designed by analyzing and calculating a frequency of each amino acid at each position in the CDRs of actual human-derived mature antibodies, then simulating sequences that reflect the calculated frequencies; and
the CDR-L3 contains 9 to 11 amino acids, the analysis of the frequencies is conducted according to each length, and the CDR-L3 sequences being designed based on an analysis result of complementarity determining region CDR-L3 of human-derived mature antibodies, which have the same amino acid lengths as CDR-L3 to be designed.

9. The method of claim 1,
Wherein, when designing light chain complementarity determining region sequences, the light chain is a kappa light chain or a lambda light chain.

10. The method of claim 1,
wherein, when designing the CDR sequences individually, for CDR-H3,
a) each sequence therefor excluding 3 amino acids from a C-terminus of the CDR are designed by reflecting a frequency of each amino acid at each position of CDRs of actual human-derived mature antibodies, and
b) 3 amino acid sequences from the C-terminus of the CDR are designed by analyzing and calculating frequencies of the 3 amino acid sequences at the corresponding positions of CDRs of actual human-derived mature antibodies, then simulating sequences that reflect the calculated frequencies; and
the CDR-H3 contains 9 to 16 amino acids, the analysis of the frequencies is conducted according to each length, and the CDR-H3 sequences being designed based on an analysis result of complementarity determining region CDR-H3 of human-derived mature antibodies, which have the same amino acid length as CDR-H3 to be designed.

11. The method of claim 1, further comprising:
after the designing of the complementarity determining region amino acid sequences,
excluding sequences with the possibility of N-glycosylation, isomerization, deamidation, cleavage and oxidation from the designed sequences.

12. The method of claim 1, further comprising:
after the designing of the complementarity determining region amino acid sequences,
deimmunizing the designed sequences.

13. The method of claim 1, further comprising:
after the designing of the complementarity determining region amino acid sequences,
reverse-translating the designed sequences into polynucleotide sequences and then designing oligonucleotide sequences in which framework region sequences of human antibody germline variable domain genes are linked to the 5′ and 3′ ends of the reverse-translated polynucleotides.

14. The method of claim 1,
wherein the antibodies include amino acid sequences encoded by IGHV3-23 (VH3-23, Genebank accession No. Z12347), IGKV3-20 (VK3-A27, Genebank accession No. X93639), IGLV1-47 (VL1g, GenBank accession No. Z73663) or fragments thereof.

15. The method of claim 1,
wherein the antibodies are selected from the group consisting of IgA, IgD, IgE, IgM, IgG, Fc fragments, Fab, Fab′, F(ab′)₂, scFv, single variable domain antibody and Fv.

16. An antibody library prepared by the method of claim 1.
